(19) United States
(12) Patent Application Publication
(10) Pub. No.: US 2012/0282246 A1
(43) Pub. Date: Nov. 8, 2012

(54) Compositions and methods for antibody and ligand identification

(76) Inventors: Roderick A. Hyde, Redmond, WA (US); Wayne R. Kindsvogel, Seattle, WA (US); Elizabeth A. Sweeney, Seattle, WA (US); Lowell L. Wood, JR., Bellevue, WA (US)

(21) Appl. No.: 13/068,302

(22) Filed: May 6, 2011

(51) Int. Cl. A61K 39/395; C12P 21/00; A61P 37/04; C12N 5/0781; A61P 31/16; C40B 30/04; C07K 16/00

(52) U.S. Cl. 424/130.1; 506/9; 435/69.6; 530/387.1; 435/377; 435/375; 435/325

(57) ABSTRACT

Embodiments disclosed herein relate to methodology, and kits thereof for identifying particular disease-associated antibodies partially based on comparative binding to a mimotope array. Isolation of identified disease-associated antibodies and uses thereof are also described.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications, including any priority claims, is incorporated herein by reference to the extent such subject matter is not inconsistent herewith.

### Related Applications

For purposes of the USPTO extra-statutory requirements, the present application constitutes a continuation-in-part of U.S. patent application Ser. No. To be Assigned, entitled COMPOSITIONS AND METHODS FOR ANTIBODY AND LIGAND IDENTIFICATION, naming Roderick A. Hyde, Wayne R. Kindsvogel, Elizabeth A. Sweeney and Lowell L. Wood, Jr. as inventors, filed 6 May 2011, which is currently co-pending, or is an application of which a currently co-pending application is entitled to the benefit of the filing date.

For purposes of the USPTO extra-statutory requirements, the present application constitutes a continuation-in-part of U.S. patent application Ser. No. To be Assigned, entitled COMPOSITIONS AND METHODS FOR ANTIBODY AND LIGAND IDENTIFICATION, naming Roderick A. Hyde, Wayne R. Kindsvogel, Elizabeth A. Sweeney and Lowell L. Wood, Jr. as inventors, filed 6 May 2011, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above, but expressly points out that such designation(s) are not to be construed in any way as any type of commentary and/or admission as to whether or not the present application contains any new matter in addition to the matter of its parent application(s).

### SUMMARY

Various embodiments are disclosed herein that relate to methods, devices, systems, and computer program products for identifying novel antibodies or novel antigens for therapeutic or diagnostic purposes. For example, disease-associated antibodies are identified from a subject by contacting at least one biological tissue of the subject with a mimotope array. In various embodiments, a comparison is made between the binding of antibodies to a mimotope array from a first subject who is afflicted or suspected of being afflicted with a disease, condition, or disorder with the binding of antibodies to a mimotope array from a second subject who is not afflicted or suspected of being afflicted with the disease, condition, or disorder of the first subject (or in an embodiment, with any disease, condition, or disorder). In an embodiment, the mimotope array is operably coupled to at least one computing device or at least part of a computing system that allows for automation or detection of at least one step of the method(s).

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates a partial view of a particular embodiment described herein.

FIG. 2 illustrates general antibody structure.

FIG. 3 illustrates a partial view of a particular embodiment described herein.

FIG. 4 illustrates a partial view of a particular embodiment described herein.

FIG. 5 illustrates a partial view of particular embodiments described herein.

FIG. 6 illustrates a partial view of particular embodiments described herein.

FIG. 7 illustrates a partial view of particular embodiments described herein.

FIG. 8 illustrates a partial view of particular embodiments described herein.

FIG. 9 illustrates a partial view of a particular embodiment described herein.

FIG. 10 illustrates a partial view of the method of FIG. 9.

FIG. 11 illustrates a partial view of the method of FIG. 9.

FIG. 12 illustrates a partial view of the method of FIG. 9.

FIG. 13 illustrates a partial view of a particular embodiment disclosed herein.

FIG. 14 illustrates a partial view of a particular embodiment disclosed herein.

FIG. 15 illustrates a partial view of a particular embodiment disclosed herein.

FIG. 16 illustrates a partial view of a particular embodiment disclosed herein.

FIG. 17 illustrates a partial view of a particular embodiment disclosed herein.

FIG. 18 illustrates a partial view of a particular embodiment disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

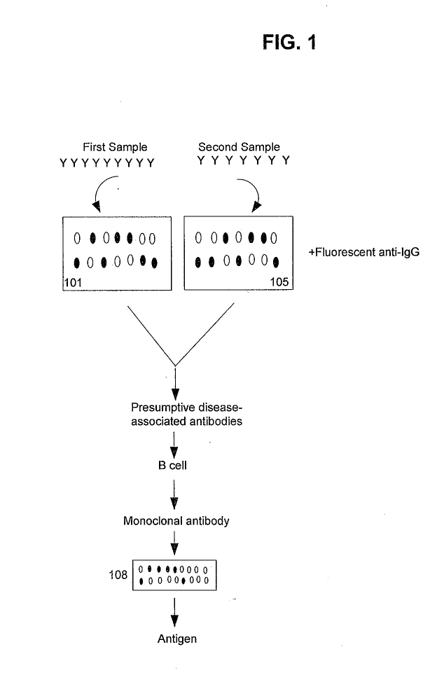

As depicted in FIG. 1, in an embodiment, a first biological sample (e.g., biological fluid such as whole blood, or serum), is contacted with a first mimotope array 101, and a second biological sample (e.g., biological fluid such as whole blood, or serum) is contacted with a second mimotope array 105. Once any antibody binding differences between the first array 101 and the second array 105 have been determined, the presumptive disease-associated antibodies are isolated. The differences in the arrays 101, 105, are determined based on antibody binding above background and based on the samples. For example, if the first sample is a normal, or healthy subject and the second sample is a diseased or vaccinated subject, etc. then the mimotopes bound by antibodies in the second sample but not the first (or at a much higher level in the second sample than in the first sample), are presumed to be disease-associated antibodies as correlative to that sample. Further, a differential screening such as this is optional, and in an embodiment, a single sample is used to identify antibodies present in the sample (e.g., healthy, recovered from disease, etc.) for therapeutic or diagnostic purposes.

In an embodiment, B cells producing presumptive disease-associated antibodies are isolated, using a mimotope array. In an embodiment, the B cells are used to clone, express, and produce disease-associated monoclonal antibodies.

In an embodiment, the presumptive disease-associated antibodies are isolated, and the corresponding B cells secreting the antibodies are isolated, as described. For example, one or more B cells can be identified, and optionally isolated, based on binding to a column containing at least one mimotope (e.g., peptoid).

In an embodiment, the multiple antibodies detected based on their ability to bind at least one mimotope, include presumptive disease-associated antibodies, which are confirmed to be disease-associated antibodies once verified (e.g., immunohistochemistry, column purification, etc.).

In an embodiment, monoclonal antibodies produced from the isolated B cells or their clones are either utilized for diagnosis or treatment of a subject (for example the subject whose biological sample was tested, or possibly a different subject entirely). Optionally, the monoclonal antibodies are further analyzed against known or unknown antigens for binding (e.g., a protein array 108), or tissue sections. Finally, by determining binding preferences for the monoclonal antibodies, antigen(s) to which the antibodies bind can be determined.

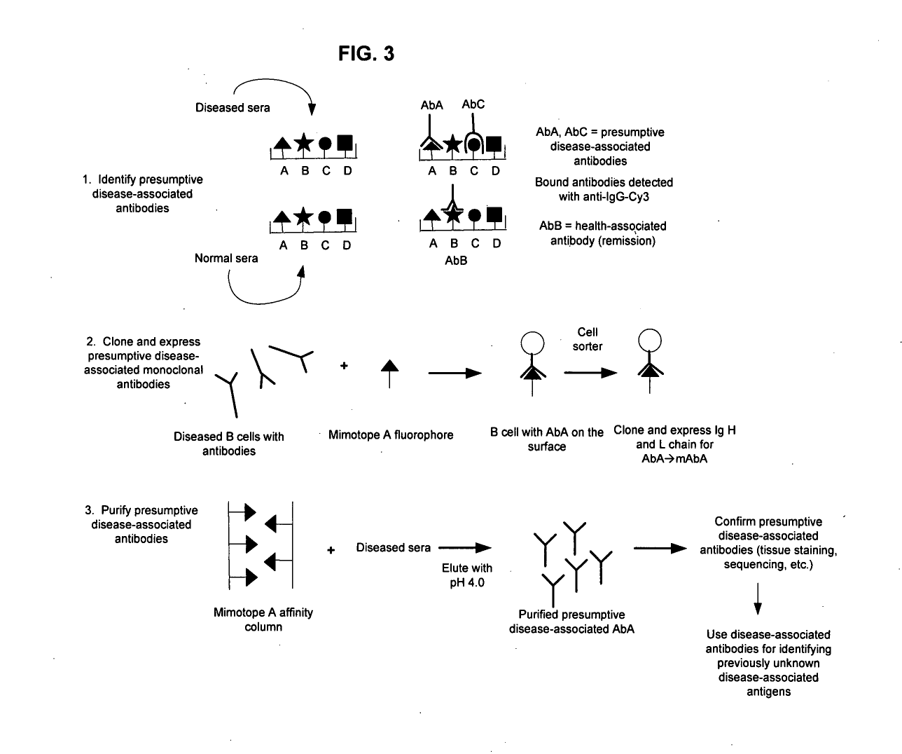

As shown in FIG. 3, in an embodiment, a method is disclosed for using one or more mimotopes (A, B, C, D) as a structure that mimics one or more epitopes (e.g., naturally occurring peptide) for identifying one or more presumptive disease-associated antibodies through binding (step 1, FIG. 3). For example, in an embodiment, an array of mimotopes is constructed, where the mimotopes have a plurality of structures. In an embodiment, the array is contacted with a biological sample of interest (e.g., from a diseased subject), and the resultant bound antibodies are analyzed.

For example, comparing the binding pattern of the biological sample of interest (e.g., serum from a diseased subject) with the binding pattern of an appropriate control sample (e.g., serum from a healthy or normal subject), bound antibodies (e.g., that are fluorescently labeled) that correspond to the biological sample of interest but not the control sample are identified as presumptive disease-associated antibodies. In an embodiment, the presumptive disease-associated antibodies are isolated (e.g., eluted). In an embodiment, the isolated presumptive disease-associated antibodies are utilized for diagnostic, prophylactic, or therapeutic use.

In an embodiment; the presumptive disease-associated antibodies are isolated, presumptive monoclonal antibodies are obtained from B cells (step 2, FIG. 3). Subsequently, in an embodiment, the isolated disease-associated antibodies and are purified and confirmed to be disease-associated antibodies (e.g., immunohistochemistry, histological staining, or other means). In an embodiment, the identified disease-associated antigens are isolated and utilized for diagnostic, prophylactic, or therapeutic use.

In an embodiment, biological sample(s) from multiple subjects may be included, for example, one or more subjects are at a particular disease state, while one or more other subjects are at a different disease state. Such cases of varying disease states (as based on symptoms, biological tests, or other means), can provide additional information regarding antigens at different stages of disease, or reduce false positive results. For example, in an embodiment, the disease-associated antibodies include antibodies produced by patients who recover from disease or other disorder during which antibodies were produced (e.g., HIV elite responders, spontaneous remission of cancer in a subject, autoimmune disease, infectious disease, etc.), can be identified and used for therapeutic or diagnostic purposes.

In an embodiment, one or more mimotopes can exhibit greater binding intensity on the array than other mimotopes. Such high level of binding is an indicator of the quantity of antibodies in the biological sample that bound the mimotope (e.g., recognizes at least a portion of the mimotope as an epitope structure). In an embodiment, isolating these antibodies when they are from the biological sample of an immunized subject can allow for utilization for passive immunization of other subjects.

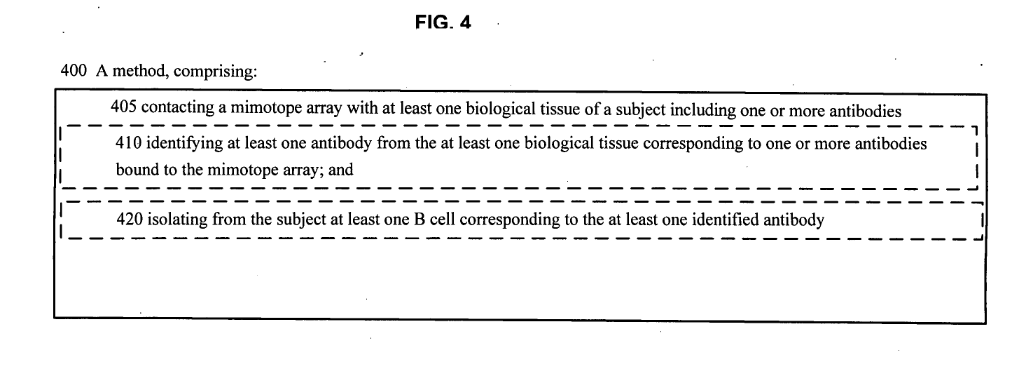

As described in FIG. 4, a method includes 405 contacting a mimotope array with at least one biological tissue of a subject including one or more antibodies, 410 identifying at least one antibody from the at least one biological tissue corresponding to one or more antibodies bound to the mimotope array; and 420 isolating from the subject at least one B cell corresponding to the at least one identified antibody.

In an embodiment, a method or system (including at least one computing device, array, other hardware or software related to the embodiment) includes identifying and optionally isolating a B cell that is responsive to at least one mimotope (e.g., peptoid). In an embodiment, the B cell responsiveness is measured as being above background noise (e.g., includes binding of a B cell receptor or antibody).

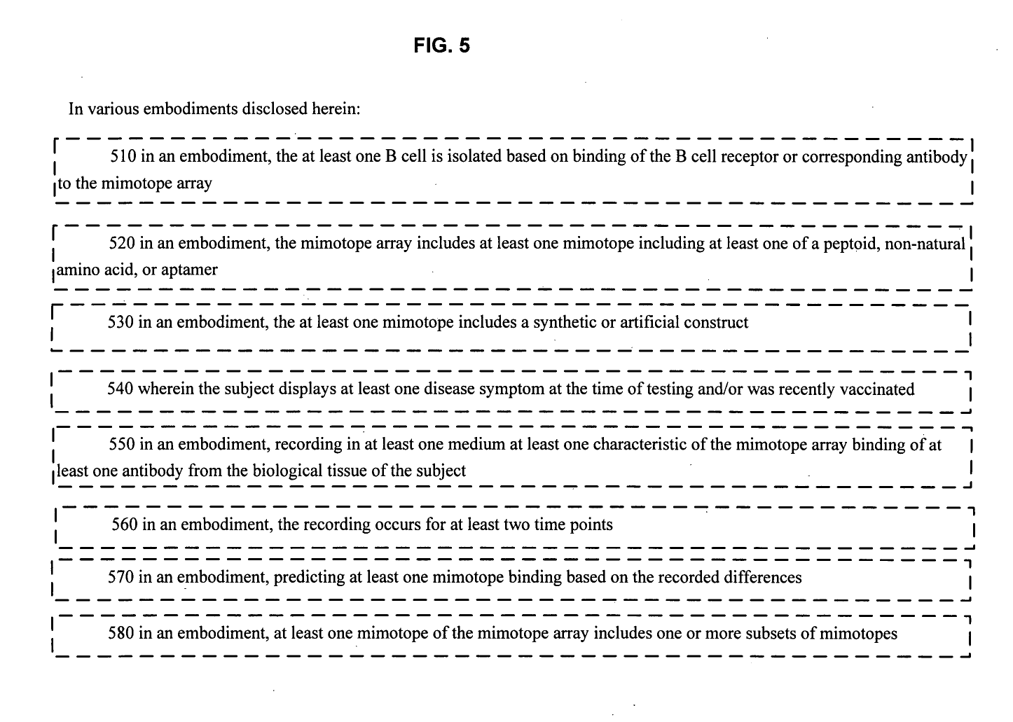

As described in FIG. 5, in an embodiment 510, at least one B cell is isolated based on binding of the B cell receptor or corresponding antibody to the mimotope array. As described elsewhere, and in FIG. 5, in an embodiment 520, the mimotope array includes at least one mimotope including at least one of a peptoid, non-natural amino acid, or aptamer. In an embodiment 530, the at least one mimotope includes a synthetic or artificial construct. In an embodiment 540, the subject displays at least one disease symptom at the time of testing and/or was recently vaccinated. In an embodiment 550, the method further comprises recording in at least one medium at least one characteristic of the mimotope array binding of at least one antibody from the biological tissue of the subject. In an embodiment 560, the recording occurs for at least two time points. In an embodiment 570, the method further comprises predicting at least one mimotope binding based on the recorded differences. In an embodiment 580, at least one mimotope of the mimotope array includes one or more subsets of mimotopes.

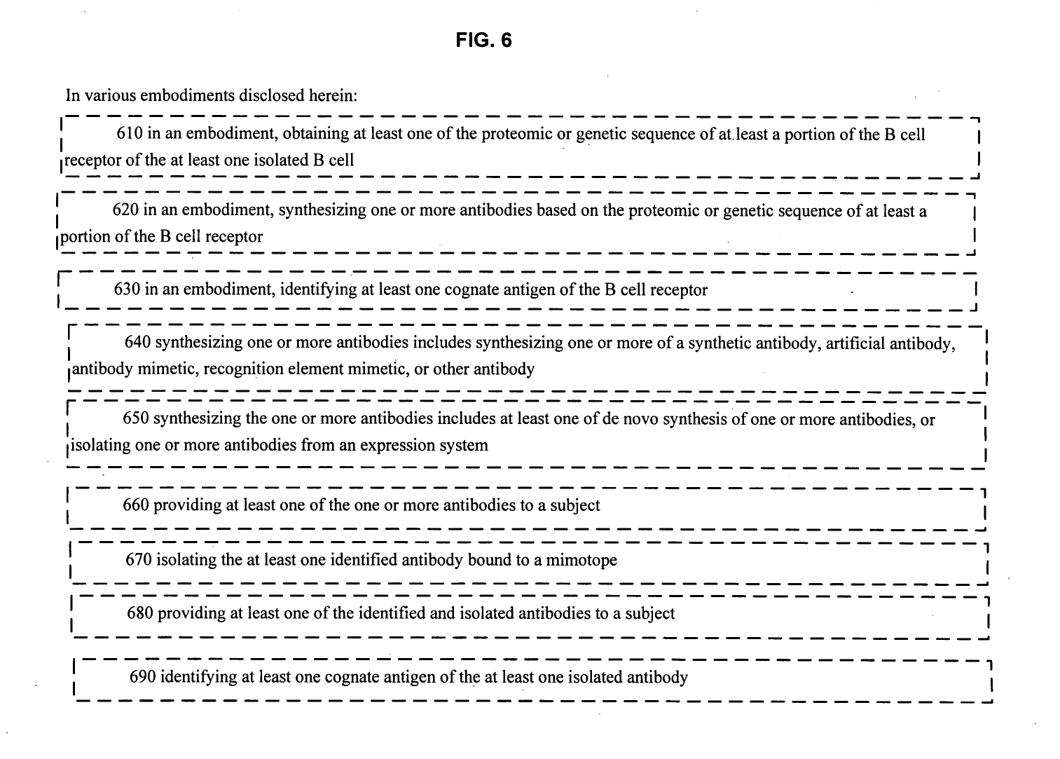

As described in FIG. 6, as well as elsewhere, in an embodiment 610, the method further comprises obtaining at least one of the proteomic or genetic sequence of at least a portion of the B cell receptor of at least one isolated B cell, isolated by way of a mimotope array. In an embodiment 620, the method further comprises synthesizing one or more antibodies based on the proteomic or genetic sequence of at least a portion of the B cell receptor. In an embodiment 630, the method further comprises identifying at least one cognate antigen of the B cell receptor. In an embodiment 640, the method further comprises synthesizing one or more antibodies including one or more of a synthetic antibody, artificial antibody, antibody mimetic, recognition element mimetic, or other antibody. In an embodiment 650, the method further comprises synthesizing one or more antibodies by at least one of de novo synthesis, or isolating one or more antibodies from an expression system. In an embodiment 660, the method further comprises providing at least one of the one or more antibodies to a subject. In an embodiment 670, the method further comprises isolating the at least one identified antibody bound to a mimotope. In an embodiment 680, the method further comprises providing at least one of the identified and isolated antibodies to a subject. In an embodiment 690, the method further comprises identifying at least one cognate antigen of the at least one isolated antibody.

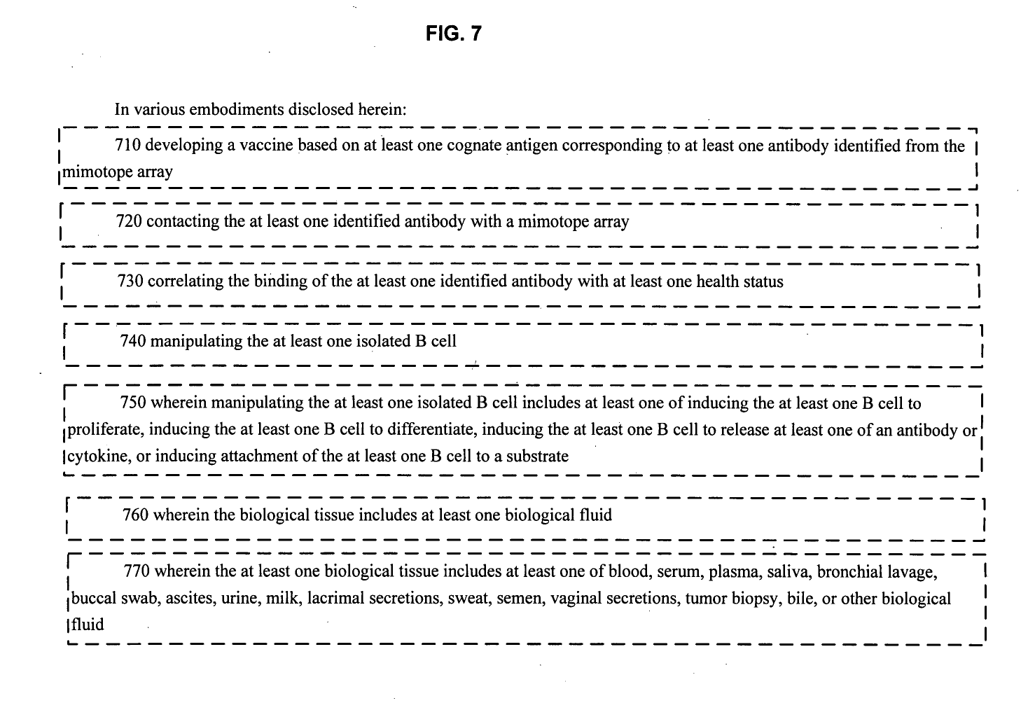

As described in FIG. 7, as well as elsewhere herein, in various embodiments, a method further comprises 710 developing a vaccine based on at least one cognate antigen corresponding to at least one antibody identified from the mimotope array. In an embodiment 720, the method further comprises contacting the at least one identified antibody with a mimotope array. In an embodiment 730, the method further comprises correlating the binding of the at least one identified antibody with at least one health status. In an embodiment 740, the method further comprises manipulating the at least one isolated B cell. In an embodiment 750, manipulating the at least one isolated B cell includes at least one of inducing the at least one B cell to proliferate, inducing the at least one B cell to differentiate, inducing the at least one B cell to release at least one of an antibody or cytokine, or inducing attachment of the at least one B cell to a substrate. In an embodiment 760, the biological tissue includes at least one biological fluid. In an embodiment 770, at least one biological tissue includes at least one of blood, serum, plasma, saliva, bronchial lavage, buccal swab, ascites, urine, milk, lacrimal secretions, sweat, semen, vaginal secretions, tumor biopsy, bile, or other biological fluid.

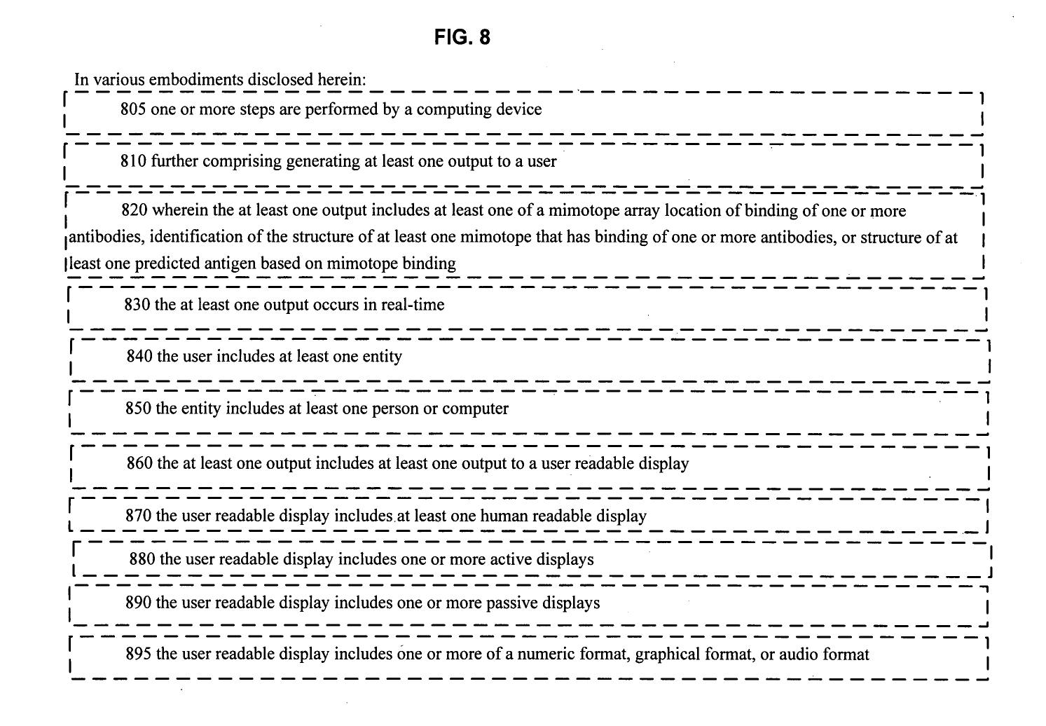

As described in FIG. 8, and elsewhere herein, in an embodiment 805, one or more steps of a method disclosed herein are performed by a computing device. In an embodiment 810, the method further comprises generating at least one output to a user. In an embodiment 820, the at least one output includes at least one of a mimotope array location of binding of one or more antibodies, identification of the structure of at least one mimotope that has binding of one or more antibodies, or the structure of at least one predicted antigen based on mimotope binding. For example, structure includes primary, secondary, or tertiary structural information. In an embodiment 830, the at least one output occurs in real-time. In an embodiment 840, the user includes at least one entity. In an embodiment 850, the entity includes at least one person or computer. In an embodiment 860, the at least one output includes at least one output to a user readable display. In an embodiment 870, the user readable display includes at least one human readable display. In an embodiment 880, the user readable display includes one or more active displays. In an embodiment 890, the user readable display includes one or more passive displays. In an embodiment 895, the user readable display includes one or more of a numeric format, graphical format, or audio format.

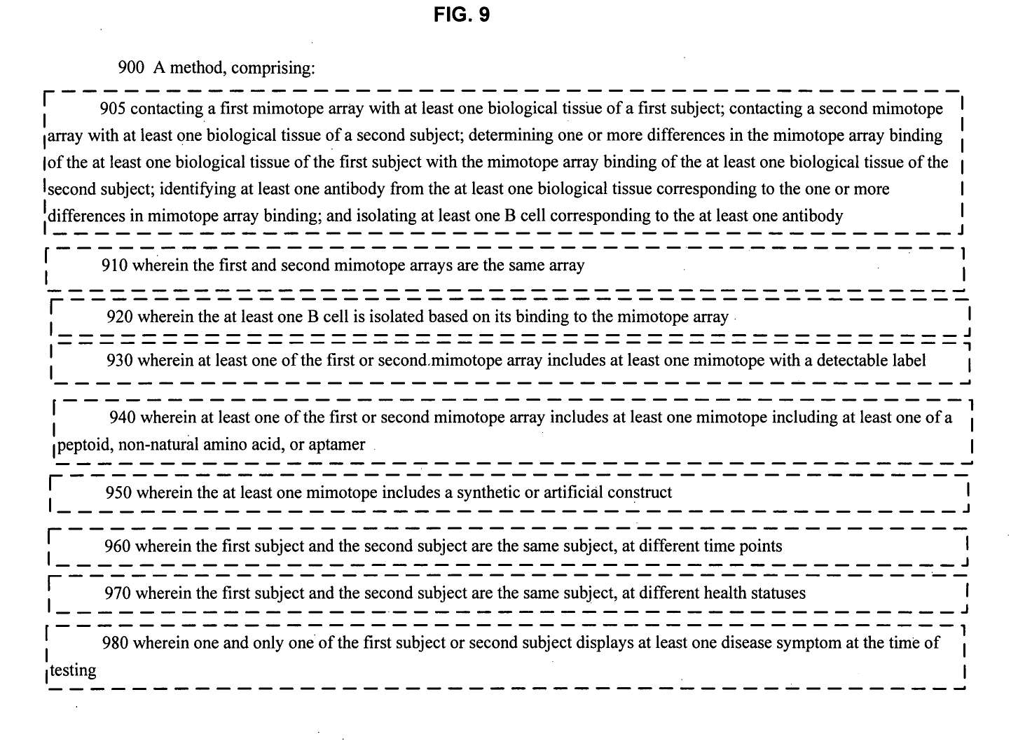

As described in FIG. 9, as well as elsewhere herein, a method 900 comprises 905 contacting a first mimotope array with at least one biological tissue of a first subject; contacting a second mimotope array with at least one biological tissue of a second subject; determining one or more differences in the mimotope array binding of the at least one biological tissue of the first subject with the mimotope array binding of the at least one biological tissue of the second subject; identifying at least one antibody from the at least one biological tissue corresponding to the one or more differences in mimotope array binding; and isolating at least one B cell corresponding to the at least one antibody. In an embodiment 910, the first and second mimotope arrays are the same array. In an embodiment 920, the at least one B cell is isolated based on its binding to the mimotope array. In an embodiment 930, at least one of the first or second mimotope array includes at least one mimotope with a detectable label. In an embodiment 940, at least one of the first or second mimotope array includes at least one mimotope including at least one of a peptoid, non-natural amino acid, or aptamer. In an embodiment 950, the at least one mimotope includes a synthetic or artificial construct. In an embodiment 960, the first subject and the second subject are the same subject, at different time points. In an embodiment 970, the first subject and the second subject are the same subject, at different health statuses. In an embodiment 980, one and only one of the first subject or second subject displays at least one disease symptom at the time of testing.

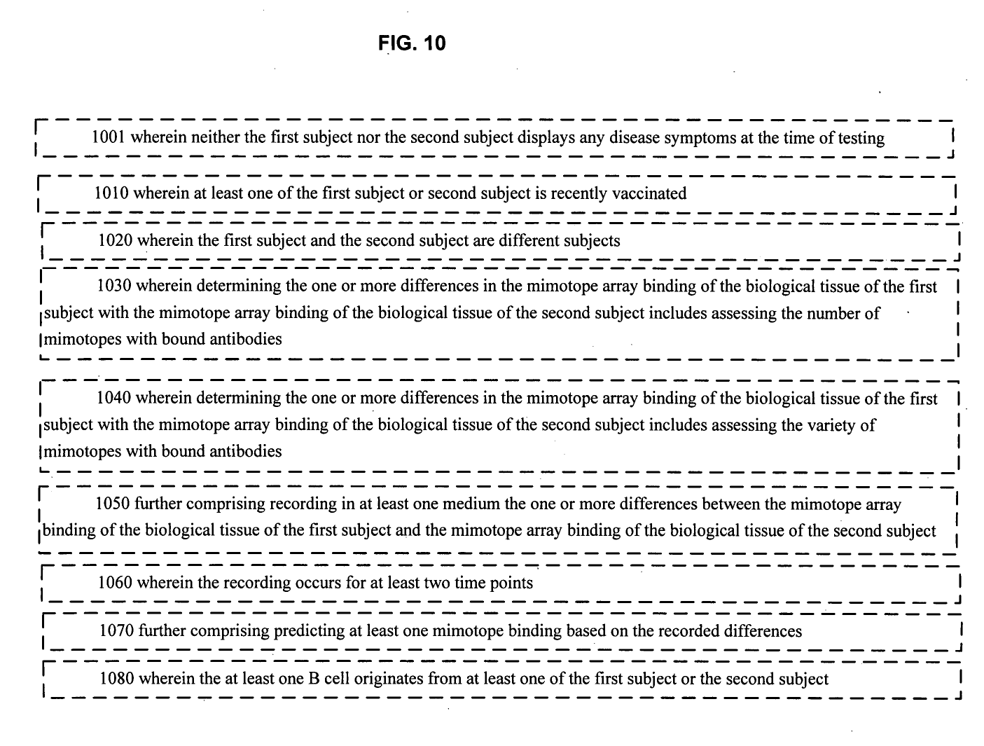

As described in FIG. 10, as well as elsewhere, in an embodiment 1001, neither the first subject nor the second subject displays any disease symptoms at the time of testing. In an embodiment 1010, at least one of the first subject or second subject is recently vaccinated. In an embodiment 1020, the first subject and the second subject are different subjects. In an embodiment 1030, determining the one or more differences in the mimotope array binding of the biological tissue of the first subject with the mimotope array binding of the biological tissue of the second subject includes assessing the number of mimotopes with bound antibodies. In an embodiment 1040, determining the one or more differences in the mimotope array binding of the biological tissue of the first subject with the mimotope array binding of the biological tissue of the second subject includes assessing the variety of mimotopes with bound antibodies. In an embodiment 1050, the method further comprises recording in at least one medium the one or more differences between the mimotope array binding of the biological tissue of the first subject and the mimotope array binding of the biological tissue of the second subject. In an embodiment 1060, the recording occurs for at least two time points. In an embodiment 1070, the method further comprises predicting at least one mimotope binding based on the recorded differences. In an embodiment 1080, the at least one B cell originates from at least one of the first subject or the second subject.

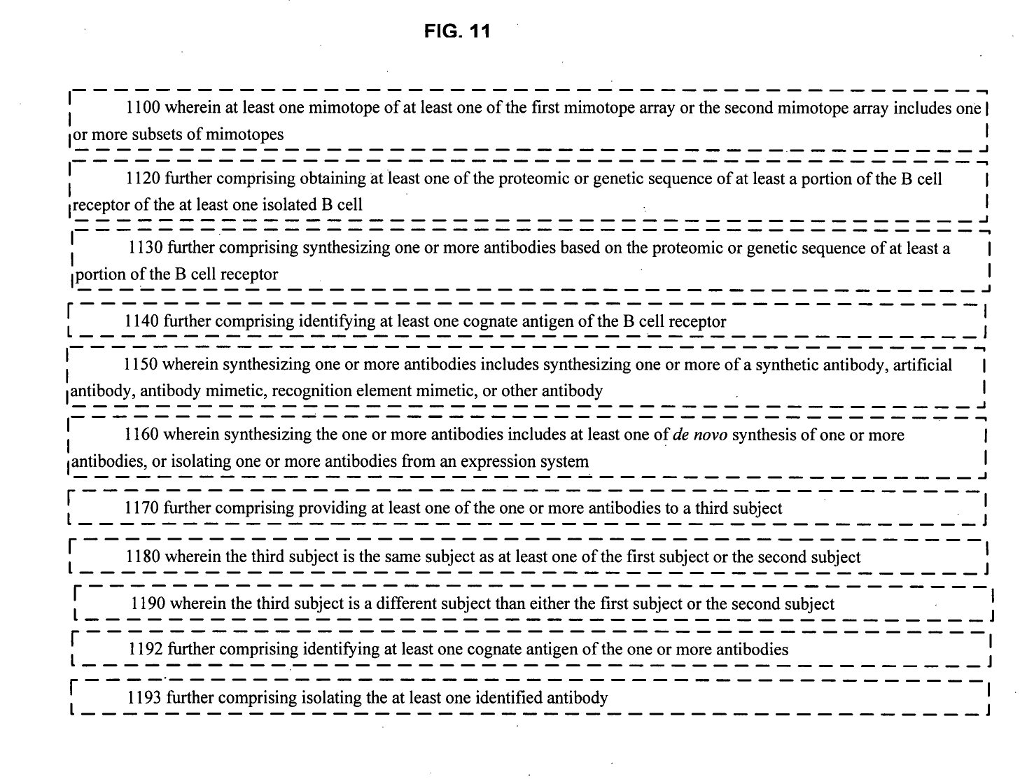

As described in FIG. 11, as well as elsewhere herein, in an embodiment 1100, at least one mimotope of at least one of the first mimotope array or the second mimotope array includes one or more subsets of mimotopes. In an embodiment 1120, the method further comprises obtaining at least one of the proteomic or genetic sequence of at least a portion of the B cell receptor of the at least one isolated B cell. In an embodiment 1130, the method further comprises synthesizing one or more antibodies based on the proteomic or genetic sequence of at least a portion of the B cell receptor. In an embodiment 1140, the method further comprises identifying at least one cognate antigen of the B cell receptor. In an embodiment 1150, the method further comprises synthesizing one or more antibodies (e.g., a synthetic antibody, artificial antibody, antibody mimetic, recognition element mimetic, or other antibody). In an embodiment 1160, synthesizing the one or more antibodies includes at least one of de novo synthesis of one or more antibodies, or isolating one or more antibodies from an expression system. In an embodiment 1170, the method further comprises providing at least one of the one or more antibodies to a third subject. In an embodiment 1180, the third subject is the same subject as at least one of the first subject or the second subject. In an embodiment 1190, the third subject is a different subject than either the first subject or the second subject. In an embodiment 1192, the method further comprises identifying at least one cognate antigen of the one or more antibodies. In an embodiment 1193, the method further comprises isolating the at least one identified antibody.

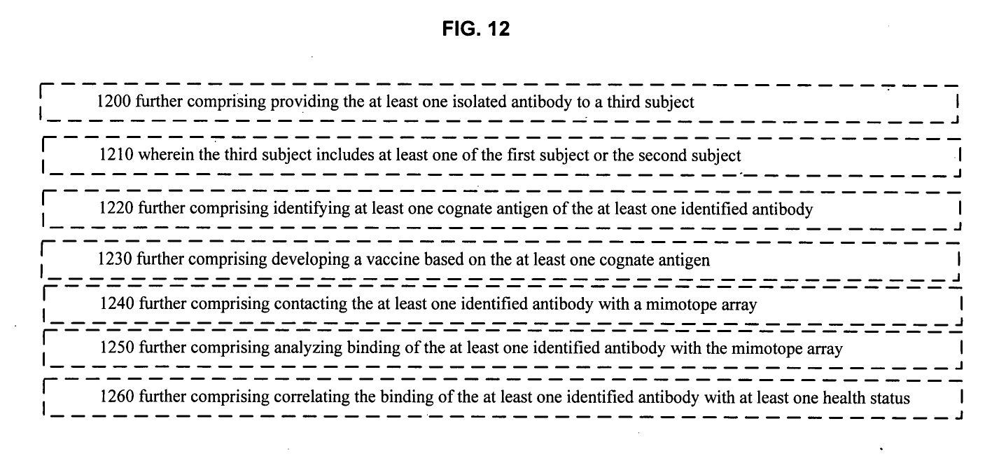

As described in FIG. 12, as well as elsewhere herein, in an embodiment 1200, the method further comprises providing the at least one isolated antibody to a third subject. In an embodiment 1210, the third subject includes at least one of the first subject or the second subject. In an embodiment 1220, the method further comprises identifying at least one cognate antigen of the at least one identified antibody. In an embodiment 1230, the method further comprises developing a vaccine based on the at least one cognate antigen. In an embodiment 1240, the method further comprises contacting the at least one identified antibody with a mimotope array. In an embodiment 1250, the method further comprises analyzing binding of the at least one identified antibody with the mimotope array. In an embodiment 1260, the method further comprises correlating the binding of the at least one identified antibody with at least one health status.

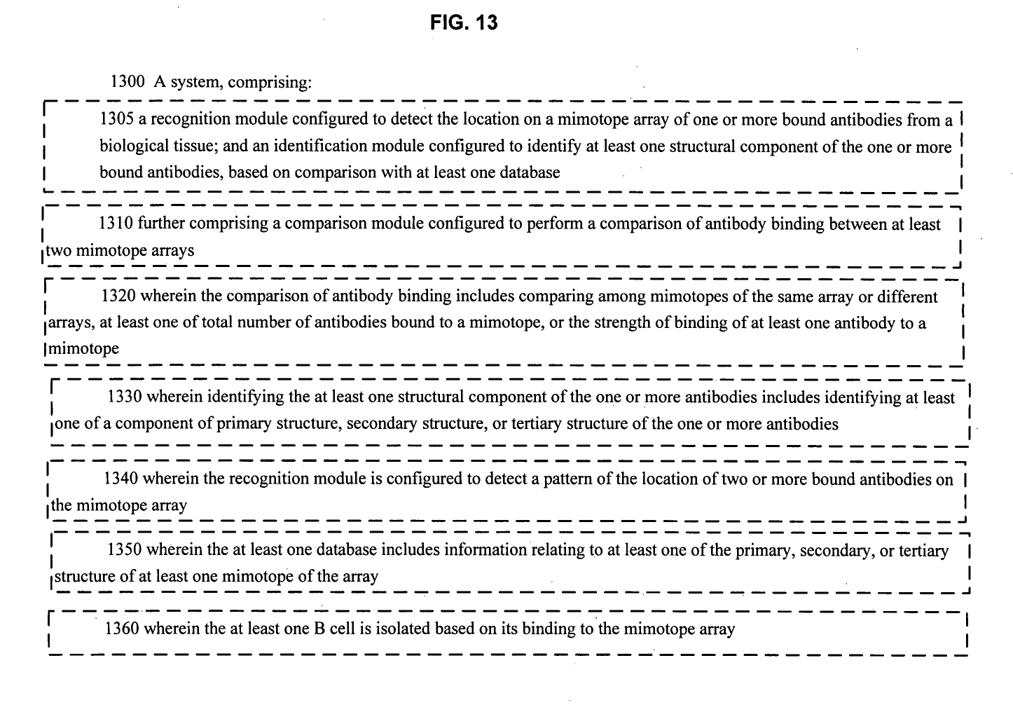

As described in FIG. 13, as well as elsewhere, a system 1300 comprises 1305 a recognition module configured to detect the location on a mimotope array of one or more bound antibodies from a biological tissue; and an identification module configured to identify at least one structural component of the one or more bound antibodies, based on comparison with at least one database. In an embodiment 1310, the system further comprises a comparison module configured to perform a comparison of antibody binding between at least two mimotope arrays. In an embodiment 1320, the comparison of antibody binding includes comparing among mimotopes of the same array or different arrays, at least one of total number of antibodies bound to a mimotope, or the strength of binding of at least one antibody to a mimotope. In an embodiment 1330, identifying the at least one structural component of the one or more antibodies includes identifying at least one of a component of primary structure, secondary structure, or tertiary structure of the one or more antibodies. In an embodiment 1340, the recognition module is configured to detect a pattern of the location of two or more bound antibodies on the mimotope array. In an embodiment 1350, the at least one database includes information relating to at least one of the primary, secondary, or tertiary structure of at least one mimotope of the array. In an embodiment 1360, the at least one B cell is isolated based on its binding to the mimotope array.

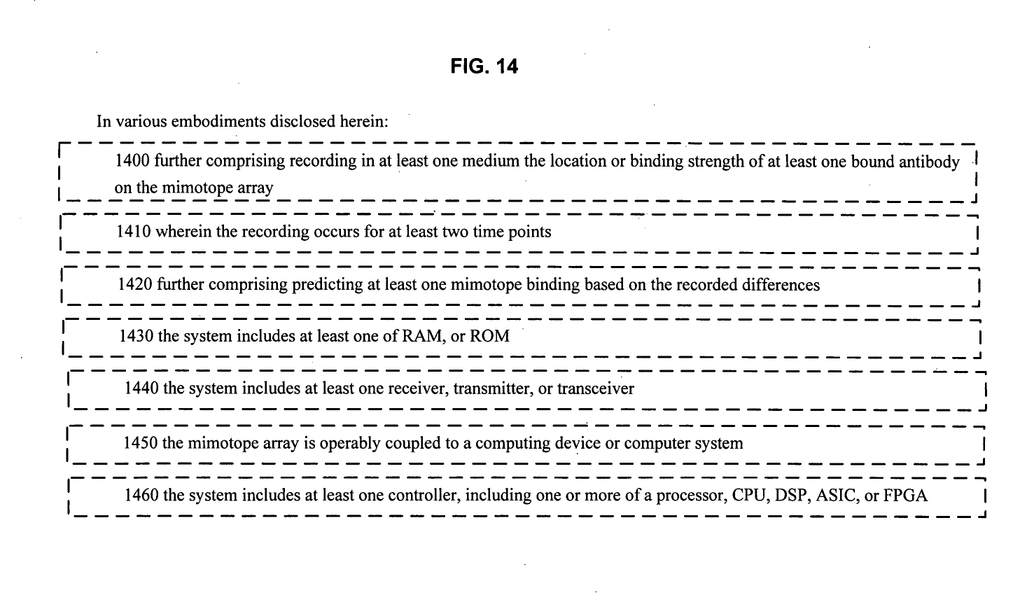

As described in FIG. 14, as well as elsewhere herein, in an embodiment 1400, the system further comprises recording in at least one medium the location or binding strength of at least one bound antibody on the mimotope array. In an embodiment 1410, the recording occurs for at least two time points. In an embodiment 1420, the system further comprises predicting at least one mimotope binding based on the recorded differences. In an embodiment 1430, the system includes at least one of RAM or ROM. In an embodiment, 1440, the system includes at least one receiver, transmitter, or transceiver. In an embodiment 1450, the mimotope array is operably coupled to a computing device or computer system. In an embodiment 1460, the system includes at least one controller, including one or more of a processor, CPU, DSP, ASIC, or FPGA.

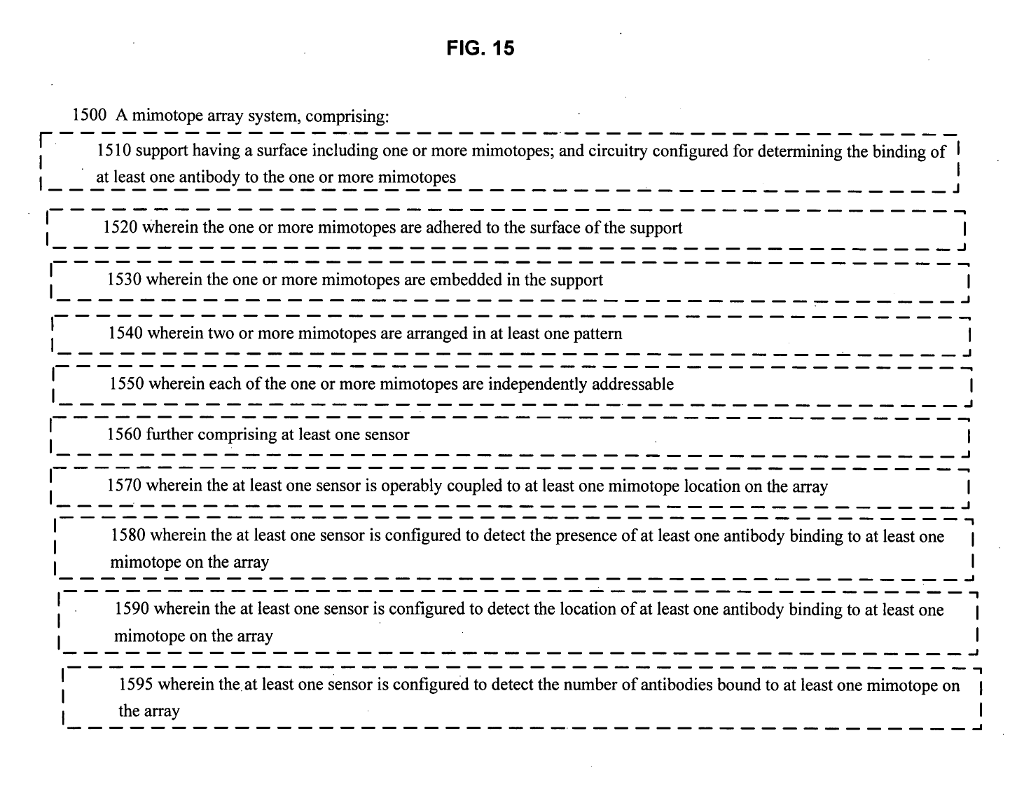

As described in FIG. 15, as well as elsewhere herein, a mimotope array system 1500, comprises in an embodiment 1510, a support having a surface including one or more mimotopes; and circuitry configured for determining the binding of at least one antibody to the one or more mimotopes. In an embodiment 1520, the one or more mimotopes are adhered to the surface of the support. In an embodiment 1530, the one or more mimotopes are embedded in the support. In an embodiment 1540, two or more mimotopes are arranged in at least one pattern. In an embodiment 1550, each of the one or more mimotopes are independently addressable. In an embodiment 1560, the system further comprises at least one sensor. In an embodiment 1570, the at least one sensor is operably coupled to at least one mimotope location on the array. In an embodiment 1580, the at least one sensor is configured to detect the presence of at least one antibody binding to at least one mimotope on the array. In an embodiment 1590, the at least one sensor is configured to detect the location of at least one antibody binding to at least one mimotope on the array. In an embodiment 1595, the at least one sensor is configured to detect the number of antibodies bound to at least one mimotope on the array.

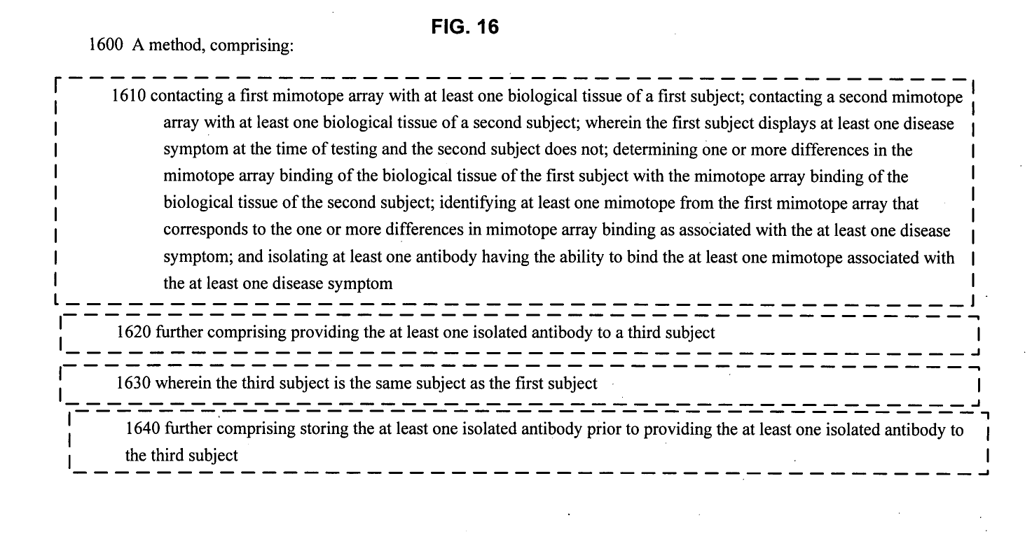

As described in FIG. 16, a method 1600, in an embodiment 1610, comprises contacting a first mimotope array with at least one biological tissue of a first subject; contacting a second mimotope array with at least one biological tissue of a second subject; wherein the first subject displays at least one disease symptom at the time of testing and the second subject does not; determining one or more differences in the mimotope array binding of the biological tissue of the first subject with the mimotope array binding of the biological tissue of the second subject; identifying at least one mimotope from the first mimotope array that corresponds to the one or more differences in mimotope array binding as associated with the at least one disease symptom; and isolating at least one antibody having the ability to bind the at least one mimotope associated with the at least one disease symptom. In an embodiment 1620, the method further comprises providing the at least one isolated antibody to a third subject. In an embodiment 1630, the third subject is the same subject as the first subject. In an embodiment 1640, the method further comprises storing the at least one isolated antibody prior to providing the at least one isolated antibody to the third subject.

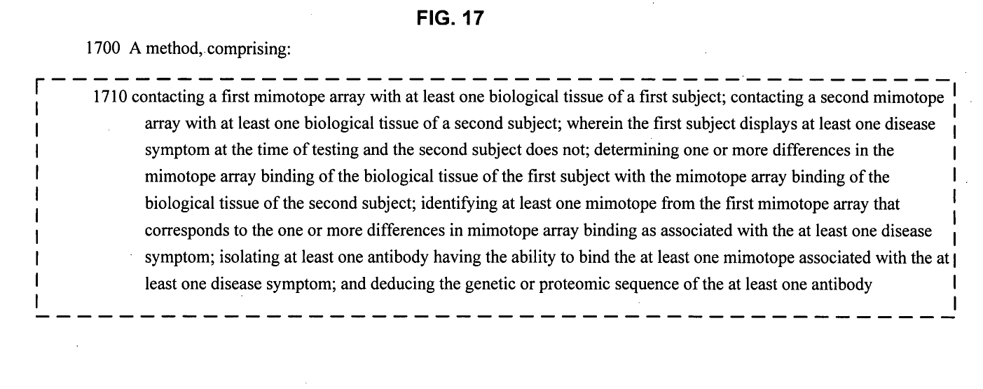

As described in FIG. 17, a method 1700, in an embodiment 1710, comprises contacting a first mimotope array with at least one biological tissue of a first subject; contacting a second mimotope array with at least one biological tissue of a second subject; wherein the first subject displays at least one disease symptom at the time of testing and the second subject does not; determining one or more differences in the mimotope array binding of the biological tissue of the first subject with the mimotope array binding of the biological tissue of the second subject; identifying at least one mimotope from the first mimotope array that corresponds to the one or more differences in mimotope array binding as associated with the at least one disease symptom; isolating at least one antibody having the ability to bind the at least one mimotope associated with the at least one disease symptom; and deducing the genetic or proteomic sequence of the at least one antibody.

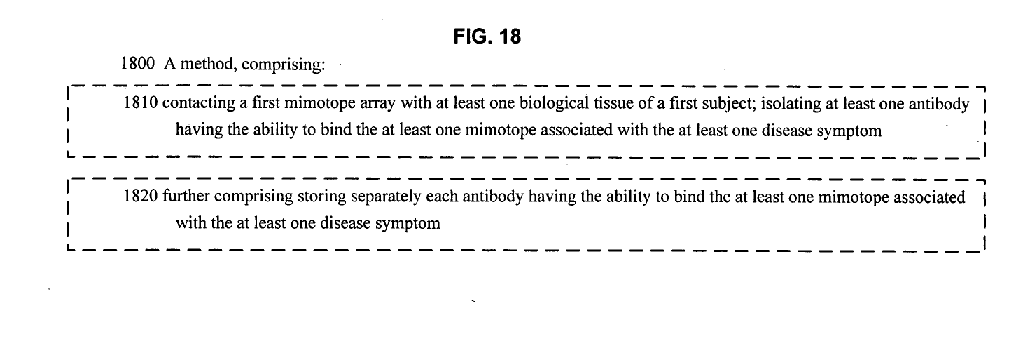

As described in FIG. 18, a method 1800, in an embodiment 1810, comprises contacting a first mimotope array with at least one biological tissue of a first subject; and isolating at least one antibody having the ability to bind at least one mimotope associated with the at least one disease symptom. In an embodiment 1820, the method further comprises storing separately each antibody having the ability to bind the at least one mimotope associated with the at least one disease symptom.

As is understood, any of the various method steps described herein are applicable to this method, as they are to any of the methods disclosed herein.

In an embodiment, the system includes one or more computer-readable media (e.g., drives, interface sockets, Universal Serial Bus (USB) ports, memory card slots, input/output components (e.g., graphical user interface, display, keyboard, keypad, trackball, joystick, touch-screen, mouse, switch, dial, etc.)).

In an embodiment, the computer-readable media is configured to accept signal-bearing media. In an embodiment, a program for causing the system to execute any of the disclosed methods can be stored on, for example, a computer-readable recording medium, a signal-bearing medium, or the like. Examples of signal-bearing media include, among others, a recordable type medium such as magnetic tape, floppy disk, hard disk drive, Compact Disc (CD), Digital Video Disk (DVD), Blu-Ray Disc, digital tape, computer memory, etc., and transmission type medium (digital and/or analog). Other non-limiting examples of signal bearing media include, for example, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, etc.

In an embodiment, one or more samples are obtained from one or more subjects at one or more time points for binding comparison, in order to ascertain disease-associated antibodies and/or antigens.

In an embodiment, the mimotope includes but is not limited to a natural, artificial, or synthetic structure that mimics an epitope. For example, the mimotope can include, but is not limited to, a non-natural peptide, peptoid, non-natural oligonucleotide, non-natural oligosaccharide, non-natural amino acid, small molecule, polymer, gel (antigen-responsive hydrogel, etc.), etc. See for example, Peppas and Huang, Pharm. Res. vol. 19, no. 5 (2002); the world wide web at: euroresidue.nl/ER_IV/Key lectures/Ye-162-173.pdf; Knappik, et al. J. Mol. Biol. vol. 296, pp. 57-86 (2000). For example, in one embodiment, synthetic polymers with molecular recognition ability are utilized for identification or isolation of disease-associated antibodies or disease-associated antigens. As another example, in an embodiment, antigen-responsive hydrogels are utilized that have a complementary antibody and antigen molecules grafted on the polymer. The collapsed gels swell when put in a buffer with corresponding free antigens that competitively bind to the antibodies, which breaks the interchain complexion and therefore allows swelling of the gel. As another example, in an embodiment, gels are formed by hybridization of oligonucleotides. See for more examples of polymers and gels as molecular recognition agents, Peppas and Huang, Id.

Synthesis of ligand libraries has been done by various methods, for example, as described in U.S. Pat. App. No. 2010/0035765, which is incorporated herein by reference. For example, the mimotope is operatively coupled to a support, such as glass, latex, plastic, a membrane, plate, bead, chip, microtiter well, etc. for binding arrays.

For example, as the diversity of the mimotope array increases, the number of epitopes that are mimicked increases exponentially. By detecting the antibodies that bind to a particular mimotope, we are able to isolate the bound antibodies (e.g., through purification column, differential array screening, or other means), obtain the genetic or proteomic (e.g., amino acid) sequence of the bound antibodies, and deduce their antigen(s) (which were previously unknown).

For example, a naturally occurring antigen includes multiple epitopes, or binding sites for one or more antibodies. Unknown antigens can be deduced and identified by working backward from the antibodies that bind to a particular mimotope structure. Furthermore, in an embodiment, isolating or cloning a B cell that secretes a monoclonal antibody identified as binding a mimotope is used to identify disease-associated antigens.

In an embodiment, the array includes one or more mimotopes at a plurality of addressable locations on the support, such that detection of binding of a particular mimotope can be cross-referenced to a known or presumptive antigen represented by the mimotope based on the location on the support.

In an embodiment, a biological sample (e.g., blood sera) is contacted with the array, and various components bind with varying affinities and specificities. In an embodiment, most components bind at an affinity and specificity that is not detectable above background noise level. However, certain components will bind with sufficient affinity and specificity for the complex to be detectable. For example, multiple antibodies will bind to the same mimotope, or antibodies will bind strongly to the same mimotope, resulting in a signal above background noise.

In an embodiment, methods of detecting bound antibodies include, but are not limited to, detecting or measuring absorbance, fluorescence, refractive index, polarization, light scattering, magnetic resonance imaging, gas phase ion spectrometry, atomic force microscopy, multipolar coupled resonance spectroscopy, nuclear magnetic resonance, or other methods.

As described, in an embodiment at least one presumptive disease-associated antibody is identified and optionally isolated. In an embodiment, the presumptive disease-associated antibodies are confirmed to be disease-associated antibodies. Depending on the amino acid sequence of the constant domain of the heavy chains (C_{H}) of the antibody, there are different isotypes: IgA, IgD, IgE, IgG, and IgM. In an embodiment, the IgG antibody can include, for example, IgG1, IgG2, IgG3, IgG4, and the IgA can include, for example, IgA1 or IgA2.

The four-chain human antibody is a heterotetrameric glycoprotein that includes two identical light (L) chains and two identical heavy (H) chains. An IgM antibody includes five heterotetramer units and an additional polypeptide (J chain). IgA antibodies are capable of forming polyvalent assemblies of two to five 4-chain units plus a J chain. An IgG antibody includes an L chain linked to an H chain by one covalent disulfide bond, while the two H chains are linked to each other by one or more disulfide bonds, depending on the H chain isotype. Each of the H and L chains has regularly spaced intrachain disulfide bridges, and each H chain has a variable region (V_{H}) at the N-terminus, followed by three constant domains (C_{H}) for each of the α and γ chains, and four C_{H} domains for μ and ε isotypes. Each L chain has a variable region (V_{L}) at the N-terminus, followed by a constant domain (C_{L}). The V_{L} is aligned with the V_{H} and the C_{L} is aligned with the first constant domain of the heavy chain (C_{H}1). The pairing of a V_{H} with a V_{L} forms a single antigen-binding site.

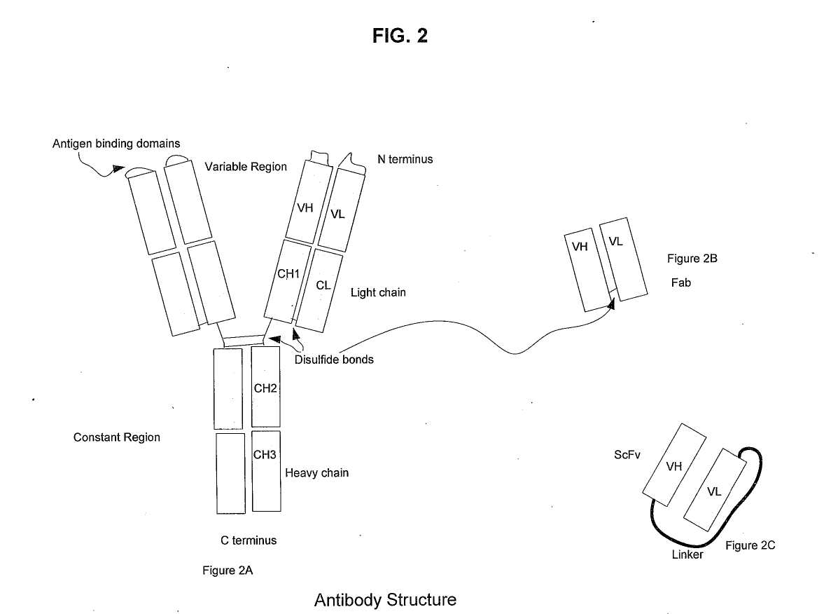

The L chain from vertebrates is categorized into one of two clearly distinct types: κ and λ, based on the amino acid sequences of the constant domains (C_{L}). For example, FIG. 2 shows an example of a generic human antibody structure (e.g., IgG, IgM, IgD, IgA, IgE). FIG. 2A shows the heavy and light chains, with the constant and variable regions of the light chain. As shown in the figure, the antigen binding domains at the N-terminus of the antibody are part of the variable region, while the Constant regions make up the heavy chain toward the C-terminus of the antibody. As shown in FIG. 2A, the heavy and light chains are joined by disulfide bonds. As shown in FIG. 2B, a Fab fragment includes a Variable heavy and Variable light chain, joined by a disulfide bond. FIG. 2C shows a Single Chain Variable Fragment (ScFv), including a Variable heavy chain, and a Variable light chain joined by a linker. Single chain antibodies are engineered to have a high binding specificity and affinity, but are smaller than monoclonal antibodies and typically have short half-lives. In an embodiment, the single chain antibody can be fused directly with a polypeptide that can be used for detection (e.g., luciferase or fluorescent proteins).

As described herein, in an embodiment a disease-associated antibody includes an antibody fragment, such as Fab, Fab′, F(ab′)₂, or Fv fragments, diabodies, linear antibodies, single-chain antibodies, or multispecific antibodies formed from antibody fragments. Various techniques are known for producing antibody fragments, for example, by using proteolytic digestion of intact antibodies, or recombinantly in host cells. In an embodiment, one or more antibodies are synthesized based on information obtained in certain embodiments, and likewise can include antibody fragments, diabodies, linear antibodies, single-chain antibodies, bispecific or multispecific antibodies formed from antibody fragments.

In an embodiment, disease-associated antibodies include bispecific or multispecific antibodies, which have binding specificities for at least two different epitopes, or multiple epitopes, respectively. In an embodiment, the at least two different epitopes are part of a single antigen. In an embodiment, the at least two different epitopes are part of different antigens. Likewise, multiple epitopes can be part of the same or different antigens.

In an embodiment, a presumptive or disease-associated antibody is isolated from the particular mimotope to which it binds by, for example, immunoaffinity purification resin columns (e.g., CH-Sepharose coupled to mimotopes). The concentration of the antibody obtained can be determined using total protein colorimetric determination, for example.

In an embodiment, one or more amino acid sequence modifications can be employed with a mimotope, for example, to improve binding affinity or other properties of the antibody. In an embodiment, one or more amino acid sequence modifications include one or more deletions, insertions, substitutions, or other chemical modification (e.g., radiolabel tagging, glycosylation, etc.). Computer algorithms have been developed to assist in choosing amino acids for modification, based on energy levels, affinity binding, or other properties.

In an embodiment, disease-associated antibodies are screened by using at least one biological sample from a subject (e.g., human patient or other animal). In an embodiment, the biological sample includes blood, serum, plasma, bronchial lavage, saliva, buccal swab, ascites, urine, milk, lacrimal secretions, sweat, semen, tumor biopsy or sample, vaginal secretions, bile, or other biological fluid.

In an embodiment, the disease, disorder, or condition includes, but is not limited to a type of cancer, a type of autoimmune disease, a virus disease, a bacterial infection, a yeast infection, a parasitic infection, a neurological disorder, a psychological condition, obesity, a blood disease, an organ disease, a metabolic disorder, pregnancy, or other disease, disorder, or condition.

In an embodiment, disease-associated antibodies are derived from the serum of a symptomatic or asymptomatic subject afflicted with or suspected of being afflicted with a disease, condition, or disorder. For example, mononuclear cells from the patient's serum containing the disease-associated antibody are used as a source for B cells, which are then cloned and induced (e.g., with cytokines, cellular receptor binding, antibodies, etc.) to become antibody-producing plasma cells. The supernatants produced by the plasma cells are screened to determine if any contains the disease-associated antibodies identified by the previous mimotope array screening, as described herein. Once a B cell clone that produces the disease-associated antibodies is identified, reverse-transcription polymerase chain reaction (RT-PCR) is performed to clone the DNAs encoding the variable regions or portions thereof of the disease-associated antibody. These sequences are then subcloned into expression vectors suitable for recombinant production of human disease-associated antibodies. The binding specificity is optionally confirmed by determining the recombinant antibody's ability to bind the disease-associated mimotopes.

In an embodiment, B cells isolated from the subject (e.g., based on expression of B cell markers, such as CD19) are plated as low as a single cell specificity per well (e.g., in a 96 well plate, 384 well plate, or 1536 well plate). The B cells are induced to differentiate into antibody-producing cells, and the culture supernatants are harvested and tested for binding to the disease-associated mimotopes.

In an embodiment, the presumptive disease-associated antibodies and potentially other antibodies that are not associated with the disease but did bind at a level greater than background, are tagged with at least one detectable label (e.g., fluorescently labeled, radiolabeled, cytotoxic drug, etc.) and FACS analysis is performed to identify the disease-associated antibodies that bind to target antigen, or target tissue. In an embodiment, target antibody binding is determined using FMAT™ analysis and instrumentation (Applied Biosystems, Foster City, Calif.). By comparing the binding of an antibody to a presumptive or known disease-associated antigen with that of a control sample (e.g., cells from a biological sample of a healthy or normal subject), the antibody is considered to preferentially bind a particular presumptive or known disease-associated antigen if the level of binding over background is at least about two-fold, at least about three-fold, at least about four-fold, at least about five-fold, at least about six-fold, at least about seven-fold, at least about eight-fold, at least about nine-fold, or at least about ten-fold, or any value therebetween or greater.

In an embodiment, polynucleotides encoding one or more of an antibody chain, variable region thereof, or fragment thereof, are isolated from cells utilizing any means available in the art. In an embodiment, polynucleotides are isolated using PCR with oligonucleotide primers that specifically bind to heavy or light chain encoding polynucleotide sequences or complements thereof using routine procedures available. For example, in an embodiment, positive samples for binding disease-associated antibodies are subjected to whole well RT-PCR to amplify the heavy and light chain variable regions of the IgG molecule expressed by the clonal plasma cells. The PCR products are then sequenced, and subcloned into human antibody expression vectors for recombinant expression in mammalian expression systems.

In an embodiment, a monoclonal antibody (MAb) is cloned and sequenced. For example, the cDNA of the MAb is isolated from a hybridoma cell line, subcloned, and expressed in mammalian cells.

In an embodiment, a full length antibody, antibody fragment, or antibody fusion protein is produced in a prokaryotic cell, or yeast cell.

In an embodiment, the detection of an antibody-antigen complex is facilitated by attaching a detectable label to the antibody. For example, suitable detectable labels include radionucleotides, enzymes, coenzymes, fluorescers, chemiluminescers, chromogens, enzyme substrates or co-factors, enzyme inhibitors, prosthetic group complexes, free radicals, particles, dyes, etc. In an embodiment, the detectable label is used for detection in an array, such as a radioimmunoassay, enzyme immunoassay (e.g., ELISA), fluorescent immunoassay, and the like.

In an embodiment, the antibodies are labeled, for example, by coupling agents (e.g., aldehydes, carbodiimides, dimaleimide, imidates, succinimides, bid-diazotized benzadine, etc.), a linker, or other method. In an embodiment, the antibodies are labeled with a therapeutic agent (e.g., a cytotoxic agent), such as a radioactive metal ion or radioisotope, abrin, ricin A, pseudomonas exotoxin, diphtheria toxin, etc.

In an embodiment, a bound antibody is detected using, for example, RIA, ELISA, precipitation, agglutination, complement fixation, immuno-fluorescence, or other procedure.

In an embodiment, the crystal structure of at least one isolated antibody is deduced and utilized to identify, for example, antigen binding sites, structural details, design for a synthetic or artificial antibody, or other purposes. Further, in an embodiment, an antigen-binding site (anti-idiotypic) information is utilized to design antigens for use in therapy (e.g., vaccines) or diagnosis. For example, once the amino acid sequence is known, tertiary structure can be assessed (e.g., extract cDNA from B cell, obtain primary sequence, then deduce the tertiary structure). In an embodiment, the tertiary structure is deduced by computer algorithm, based on the amino acid sequence. In an embodiment, NMR or X-ray crystallography is used to deduce the tertiary structure.

In an embodiment, the isolated antibody is used as a template for antibody binding (e.g., anti-idiotypic monoclonal antibody found in a monoclonal antibody expression system, or a mimetic designed to bind the binding region of the monoclonal antibody). In an embodiment, the template includes a mimetic designed to bind to the binding region of the antibody, and can be utilized, for example, in a monoclonal antibody expression system to identify a monoclonal antibody corresponding to the isolated antibodies.

In an embodiment, disease-associated antibodies described herein differentiate between a symptomatic or asymptomatic subject infected, afflicted, or suspected of being afflicted with a particular disease, condition, or disorder (“diseased subject”) and a subject that is normal, unafflicted with the particular disease, condition, or disorder (“healthy subject”). For example, an increased amount of antibody bound to the diseased subject sample compared to the healthy subject (control) sample, indicates the presence of abnormal immunity (e.g., infected cells, inflammation, or auto-immune disease cells, etc.) in the diseased subject sample. Optionally, a biological sample obtained from a diseased subject is contacted with disease-associated antibodies for a time and under conditions sufficient to allow the disease-associated antibodies to bind to cells. Bound antibody is then detected, and the presence of bound antibody indicates that the sample contains infected cells (this is helpful, for example, if the disease-associated antibodies do not bind healthy subject cells at a detectable level). Disease-associated antibodies are also useful for determining binding specificities to various strains of the disease (e.g., virus).

In an embodiment, kits useful in identifying disease-associated antibodies, or utilizing the isolated or identified disease-associated antibodies for diagnostic, prophylactic, or treatment purposes are included. In an embodiment, the disease-associated antibodies are therapeutically effective themselves. In an embodiment, the disease-associated antibodies are joined with a therapeutic agent for therapeutic efficacy. In an embodiment, the disease-associated antibodies are used to identify previously unknown disease-associated antigens.

In an embodiment, for in vivo treatment of human or other subjects, the disease-associated antibodies are administered as a pharmaceutical formulation. In an embodiment, the disease-associated antibody formulation is administered, for example, by intravenous, intramuscular, intraperitoneal, intracerobrospinal, subcutaneous, intra-articular, intrasynovial, intrathecal, oral, topical, inhalation, or other mode. In an embodiment, the disease-associated antibodies are administered locally, systemically, or parenterally.

In an embodiment, the disease-associated antibodies are formulated with pharmaceutical vehicles (e.g., oils, ethyl oleate, liposomes, etc.), and formulated at concentrations of, for example, about 1 μg/ml, about 5 μg/ml, about 10 μg/ml, about 20 μg/ml, about 100 μg/ml, about 500 μg/ml, about 1 mg/ml, about 5 mg/ml, about 10 mg/ml, or any value therebetween or greater. The dose and dosage regimen depends on a variety of factors, readily determined by a physician, including but not limited to the nature of the disease, disorder (e.g., infection) or condition, the characteristics of the disease-associated antibodies, the properties of any therapeutic agent included in the formulation, therapeutic index, the subject's overall health, and the subject's history. Generally, a therapeutically effective amount of a disease-associated antibody formulation is administered to a subject, and the subject's progress is monitored. In an embodiment, other therapeutic regimens are combined with the administration of the disease-associated antibodies, including co-administration using separate formulations or a single formulation, administered in sequence or simultaneously.

In an embodiment, the method or system further comprises recording in at least one medium the one or more differences between the mimotope array binding of the biological tissue of the first subject and the mimotope array binding of the biological tissue of the second subject. In an embodiment, the recording occurs for at least two time points. In an embodiment, the method further comprises predicting at least mimotope binding based on the recorded differences. In an embodiment, a mimotope array is developed with predicted antibody mutation (e.g., by use of a computer algorithm) based on past mutation(s) and, optionally, any known antigens associated with the antibody (e.g., antigens associated with pathogen(s) or auto-antigens, either from which antibodies have been identified or isolated).

In an embodiment, an anti-idiotype is utilized for therapeutic or preventative treatment (e.g., vaccine).

As described in the Prophetic Examples herein, disease-associated antibodies of particular embodiments can be produced in a number of ways. For example, human antibodies can be generated in vitro by activated B cells, or in transgenic animals (e.g., mice) that produce a full repertoire of antibodies in the absence of endogenous immunoglobulin production. (See for example, WO 2010/107939, which is incorporated herein by reference.)

In particular embodiments described herein the disease-associated antibodies are chimeric antibodies that include sequences derived from both human and non-human sources. For example, the chimeric antibodies can be humanized or primatized.

For example, in an embodiment, Ig genes encoding monoclonal antibodies associated with a particular disease (e.g., infection), disorder, or condition, are obtained from diseased patients using unknown antigen arrays. Rapid identification, isolation and cloning of the disease-associated Immunoglobulin genes allow production of monoclonal antibodies useful for passive immunization of individuals afflicted by, suspected of being afflicted by, or at risk for developing the particular disease, disorder, or condition.

Disease-associated antibodies are identified using arrays of mimotopes (e.g., unknown or known antigens). For example, libraries of peptoid antigens (e.g., N-substituted oligoglycines) are constructed that contain, for example, greater than about 1,000,000 different peptoid antigens. In an embodiment, the array includes at least about 1,000, at least about 2,000, at least about 3,000, at least about 4,000, at least about 5,000, at least about 6,000, at least about 7,000, at least about 8,000, at least about 9,000, at least about 10,000, at least about 12,000, at least about 20,000, at least about 50,000, at least about 100,000, at least about 500,000, at least about 1,000,000, or any value therebetween, or greater, distinct chemical species or random mimotopes. In an embodiment, an array includes, but is not limited to, a support such as a glass slide, plate, chip, bead, or any combination thereof. In an embodiment, the mimotope is cross-linked with a binding moiety to a support to form the array. In an embodiment, each of the mimotopes is an unknown antigen. In an embodiment, at least one mimotope is a known antigen. In an embodiment, a known antigen serves as a control for the binding reaction. Thus, binding level can be detected and analyzed in order to determine whether the binding reaction is specific, or above background noise levels.

Individual peptoids with a terminal cysteine residue are placed in wells of a microtiter plate (see e.g., U.S. Patent Application No. 2010/0303805, which is incorporated herein by reference), and replicate peptoid antigen arrays with individual peptoids at defined locations are printed onto maleimide-coated glass slides. For example, arrays with approximately 15,000 different octameric peptoids are tested for binding to serum-derived antibodies from normal, healthy volunteers or (symptomatic or asymptomatic) diseased patients. Methods to screen peptoid arrays with sera and recover disease-associated peptoid antigens are described (see e.g., Reddy et al., Cell 144: 132-142, 2011 which is incorporated herein by reference). Antibodies bound to peptoids on the array are detected with fluorescently labeled anti-Ig antibodies (e.g., Alexa-647 labeled anti-human-IgG antibody available from Invitrogen, Carlsbad, Calif.). Arrays with bound antibodies are analyzed with a scanner at 10 μm resolution (e.g., GenePix Autoloader 4200AL Scanner available from Molecular Devices, Sunnyvale, Calif.) and scanned images are analyzed with software (e.g., GenePix Pro 6.0 available from Axon Instruments, Union City, Calif.). Peptoid antigens on the array that bind to antibodies from diseased subject sera but not to antibodies from healthy subject sera are identified as disease-associated peptoid antigens, and the bound antibodies are identified as disease-associated antibodies. Disease-associated peptoid antigens are recovered from the array, analyzed, and used to clone and express disease-associated monoclonal antibodies. Differential screening strategies using the peptoid antigen array are designed to preferentially identify valuable antibodies for therapy. Monoclonal antibodies that recognize multiple subtypes of the disease, condition, or disorder are described (see e.g., Ekiert et al., Science 324: 246-251, 2009, which is incorporated by reference herein). For example, sera from individuals known to have recovered from different strains of influenza virus (e.g., H1N1, H3N2, and H5N1), or HIV are used to identify antibodies that recognize the same peptoid antigen, and the peptoid antigen is used to identify, clone and express an antibody useful for treating multiple strains of the virus, respectively.

To produce disease-associated monoclonal antibodies for therapy or prophylaxis, the corresponding disease-associated peptoid antigens are used to isolate B cells expressing the antibodies, and the corresponding Ig genes are amplified, cloned and expressed. Peptoid antigens identified using diseased subject sera as described above are recovered from the array and their mass is determined using tandem mass spectrometry. Methods and instrumentation for mass spectrometry are available from Bruker Daltonics Inc., Billerica, Mass. Disease-associated soluble peptoids are resynthesized in microgram quantities using a peptide synthesizer (e.g., ABI 433A Peptide Synthesizer available from Applied Biosystems Inc., Foster City, Calif.), and the submonomer method (see e.g., U.S. Patent Application No. 2010/0303805). The peptoids are purified by reverse phase-high pressure liquid chromatography on C18 columns (chromatography systems are available from Waters Corp., Milford, Mass.). The resynthesized, purified peptoid antigens are optionally reanalyzed by mass spectrometry to verify mass, and then tested for binding to disease-associated antibodies, as described above. The disease-associated peptoid antigen is used to create a probe for B cells producing disease-associated antibodies.

The verified, disease-associated peptoid is labeled with a fluorescent tag, and used to stain and sort cognate B cells obtained from the peripheral blood of diseased subjects displaying disease-associated antibodies in their serum. A fluorescent tag (e.g., Alexa-594®) is conjugated to the purified peptoid by covalent attachment to a sulfhydryl group on the peptoid. A kit including reagents and methods for conjugating Alexa Fluor® 594 C5 maleimide to peptides is available from Invitrogen, Carlsbad, Calif. (see Invitrogen Document: “Thiol-Reactive Probes,” which is incorporated herein by reference). The fluorescently labeled peptoid is then used to bind cognate B cells obtained from the peripheral blood of disease subjects, and individual B cells stained with fluorescent peptoid antigen are sorted into wells of a microtiter plate using a flow cytometer. Peripheral blood mononuclear cells are prepared from diseased subject's blood, and B cells are enriched using anti-human IgG conjugated magnetic microbeads (Magnetic beads, antibodies and protocols are available from Miltenyi Biotec, Bergisch Gladbach, Germany.). Prior to single cell sorting, IgG-positive B cells are bound with Alexa-594-peptoid (see e.g., U.S. Patent Appl. No. 2010/0303835 Ibid.), and stained with anti-CD19-APC. Single cell sorting using a FACSVantage cell sorter (available from Becton Dickinson, Palo Alto, Calif.) collects individual B cells in the wells of a microtiter plate containing RNA lysis buffer. Methods for single cell sorting of B cells are described (see e.g., Wardemann et al., Science 301: 1374-1377, 2003, and Wrammert et al., Nature 453: 667-671, 2008; each of which is incorporated herein by reference). Immunoglobulin mRNA for Ig heavy and Immunoglobulin light chains are amplified by RT-PCR and the respective DNA sequences are determined. Molecular cloning and expression of monoclonal antibodies is done as described (see e.g., Wrammert et al., Ibid.), and the recombinant monoclonal antibodies are tested for binding to the disease-associated peptoid on an array as described above. Disease-associated monoclonal antibodies are tested for binding to various strains or subtypes in an enzyme-linked immunosorbent array (ELISA) that uses purified antigens which are coated onto microtiter plates (e.g., ELISA). Disease associated monoclonal antibodies are tested for antigen neutralization and to assess their specificity and function. Methods to measure antigen neutralization are described (e.g., WO 2010/107939 which is incorporated herein by reference).

In an embodiment, an antigen identified by the process comprising: contacting a first mimotope array with at least one biological tissue of a first subject; contacting a second mimotope array with at least one biological tissue of a second subject; determining one or more differences in the mimotope array binding of the at least one biological tissue of the first subject with the mimotope array binding of the at least one biological tissue of the second subject; identifying at least one antibody from at least one biological tissue corresponding to the one or more differences in mimotope array binding; and isolating at least one antibody corresponding to at least one of the one or more differences in mimotope array binding; and contacting the at least one isolated antibody with an array including at least one unknown antigen; determining binding of the at least one isolated antibody with the at least one unknown antigen. In an embodiment, the determining binding of the at least one isolated antibody with the at least one unknown antigen includes obtaining the at least one isolated antibody bound to the at least one unknown antigen, and contacting the isolated antibody with tissue specific antigens; and measuring binding. For example, in an embodiment, the bound antibodies are contacted with tissue sections (e.g., immunohistochemistry), cell lines, or proteomic array(s), in order to determine antigen(s).

Any of the compositions described herein are optionally included in a kit. For example, one or more mimotopes, support(s), buffer(s), linker(s), other reagents, control antibodies, etc. are included in a kit embodiment. In an embodiment, a kit includes test samples, detection labels, or chemicals related to processing or detection of bound antibodies. In an embodiment, the kit is packaged (for example in a lyophilized or aqueous form), and optionally include at least one container (such as a vial, test tube, flask, bottle, support, syringe, etc.), and optionally include written instructions.

### Various Non-Limiting Embodiments are Described Herein as Prophetic Examples

### PROPHETIC EXAMPLE 1

### Molecular Cloning of Monoclonal Antibodies (Monoclonal Antibodies) for Passive Immunization to Influenza Virus

Rapid identification, isolation and cloning of influenza-associated Immunoglobulin genes allow production of monoclonal antibodies useful for passive immunization of individuals infected by influenza virus or at risk of infection by influenza virus. Immunoglobulin genes encoding antibodies associated with influenza virus infection are obtained from diseased patients using unknown antigen arrays.

Influenza virus-associated antibodies are identified from the sera of diseased influenza patients using an array of unknown antigens. Patients who have recovered from infection by influenza virus are a source of antibodies which are useful for therapy of influenza viral infections (see e.g., Khurana et al., PLoS Med. 6: e1000049, 2009, which is incorporated herein by reference). Influenza disease-associated antibodies are identified using arrays of unknown antigens. For example, libraries of peptoid antigens (e.g., N-substituted oligoglycines) are constructed that contain greater than 100,000 different peptoid antigens. Individual peptoids with a terminal cysteine residue are placed in wells of a microtiter plate (see e.g., U.S. Patent Application No. 2010/0303805, which is incorporated herein by reference), and replicate peptoid antigen arrays with individual peptoids at defined locations are printed onto maleimide-coated glass slides. For example, arrays with approximately 15,000 different octameric peptoids are tested for binding to serum-derived antibodies from normal, healthy volunteers or (symptomatic or asymptomatic) influenza patients. Methods to screen peptoid arrays with sera and recover disease-associated peptoid antigens are described (see e.g., Reddy et al., Cell 144: 132-142, 2011 which is incorporated herein by reference). Antibodies bound to peptoids on the array are detected with fluorescently labeled anti-Ig antibodies (e.g., Alexa-647 labeled anti-human-IgG antibody available from Invitrogen, Carlsbad, Calif.). Arrays with bound antibodies are analyzed with a scanner at 10 μm resolution (e.g., GenePix Autoloader 4200AL Scanner available from Molecular Devices, Sunnyvale, Calif.) and scanned images are analyzed with software (e.g., GenePix Pro 6.0 available from Axon Instruments, Union City, Calif.). Peptoid antigens on the array that bind to antibodies from influenza patient sera but not to antibodies from healthy donor sera are identified as influenza virus disease-associated peptoid antigens, and the bound antibodies are identified as disease-associated antibodies. Disease-associated peptoid antigens are recovered from the array, analyzed, and used to clone and express disease-associated monoclonal antibodies. Differential screening strategies using the peptoid antigen array are designed to preferentially identify valuable antibodies for therapy. Monoclonal antibodies that recognize multiple subtypes of influenza virus are described (see e.g., Ekiert et al., Science 324: 246-251, 2009, which is incorporated by reference herein). For example, sera from individuals known to have recovered from different strains of influenza virus (e.g., H1N1, H3N2, and H5N1) are used to identify antibodies that recognize the same peptoid antigen, and the peptoid antigen are used to clone and express a monoclonal antibody useful for treating multiple strains of influenza.

To produce disease-associated monoclonal antibodies for therapy or prophylaxis of influenza virus infections, the corresponding disease-associated peptoid antigens are used to isolate B cells expressing the desired antibodies, and the corresponding Ig genes are amplified, cloned and expressed. Peptoid antigens identified using influenza patient sera as described above are recovered from the array and their mass is determined using tandem mass spectrometry. Methods and instrumentation for mass spectrometry are available from Bruker Daltonics Inc., Billerica, Mass. Disease-associated soluble peptoids are resynthesized in microgram quantities using a peptide synthesizer (e.g., ABI 433A Peptide Synthesizer available from Applied Biosystems Inc., Foster City, Calif.), and the submonomer method (see e.g., U.S. Patent Application No. 2010/0303805). The peptoids are purified by reverse phase-high pressure liquid chromatography on C18 columns (chromatography systems are available from Waters Corp., Milford, Mass.). The resynthesized, purified peptoid antigens are optionally reanalyzed by mass spectrometry to verify mass, and then tested for binding to disease-associated antibodies, as described above. The disease-associated peptoid antigen is used to create a probe for B cells producing disease-associated antibodies.

The verified, influenza disease-associated peptoid is labeled with a fluorescent tag, and used to stain and sort cognate B cells obtained from the peripheral blood of influenza patients displaying influenza disease-associated antibodies in their serum. A fluorescent tag (e.g., Alexa-594®) is conjugated to the purified peptoid by covalent attachment to a sulfhydryl group on the peptoid. A kit including reagents and methods for conjugating Alexa Fluor® 594 C5 maleimide to peptides is available from Invitrogen, Carlsbad, Calif. (see Invitrogen Document: “Thiol-Reactive Probes,” which is incorporated herein by reference). The fluorescently labeled peptoid is then used to bind cognate B cells obtained from the peripheral blood of influenza patients, and individual B cells stained with fluorescent peptoid antigens are sorted into wells of a microtiter plate using a flow cytometer. Peripheral blood mononuclear cells are prepared from influenza patient blood, and B cells are enriched using anti-human IgG conjugated magnetic microbeads (Magnetic beads, antibodies and protocols are available from Miltenyi Biotec, Bergisch Gladbach, Germany). Prior to single cell sorting, IgG-positive B cells are bound with Alexa-594-peptoid (see e.g., U.S. Patent Appl. No. 2010/0303835 Ibid.), and stained with anti-CD19-APC. Single cell sorting using a FACSVantage cell sorter (available from Becton Dickinson, Palo Alto, Calif.) collects individual B cells in the wells of a microtiter plate containing RNA lysis buffer. Methods for single cell sorting of B cells are described (see e.g., Wardemann et al., Science 301: 1374-1377, 2003, and Wrammert et al., Nature 453: 667-671, 2008; each of which is incorporated herein by reference). Immunoglobulin mRNA for Ig heavy and Ig light chains are amplified by RT-PCR and the respective DNA sequences are determined. Molecular cloning and expression of the influenza disease-associated monoclonal antibodies is done as described (see e.g., Wrammert et al., Ibid.), and the recombinant monoclonal antibodies are tested for binding to the disease-associated peptoid on an array as described above. Influenza virus disease-associated monoclonal antibodies are tested for binding to influenza virus subtypes in an enzyme-linked immunosorbent array (ELISA) that uses purified influenza virions which are coated onto microliter plates. For example, influenza virus strains: A/New Caledonia/20/99 (H1N1), A/California/7/2/2004 (H3N2) and A/Vietnam/1203/2004 (H5N1) are adsorbed to separate plates and the influenza disease-associated monoclonal antibodies are applied, washed and detected using anti-Immunoglobulin reagents. Influenza virus ELISAs are described (see e.g., Wrammert et al, Ibid.) which allow determination of MAb specificity and affinity for influenza virus strains. True influenza virus-disease associated monoclonal antibodies are tested for virus neutralization and to assess their specificity and function. Methods to measure virus neutralization are described (e.g., WO 2010/107939 which is incorporated herein by reference).

### PROPHETIC EXAMPLE 2

### Molecular Cloning and Expression of Prostate Cancer Disease-Associated Monoclonal Antibodies Using A Peptoid Array

Prostate cancer patients may have circulating antibodies that are beneficial for treatment of their disease. Peptoid arrays displaying unknown antigens are used to identify presumptive prostate cancer disease-associated antibodies and to isolate disease-associated B cells. Immunoglobulin genes encoding presumptive disease-associated monoclonal antibodies are cloned and expressed from the B cells and used for therapy and target identification in prostate cancer.

Antibodies arising in the sera of prostate cancer patients are identified using peptoid arrays and differential screening with sera from healthy controls, and prostate cancer patients, respectively. Sera containing antibodies associated with prostate cancer (see e.g., Wang et al., New Engl. J. Med. 353: 1224-1235, 2005, which is incorporated herein by reference) are tested on peptoid arrays. Individual peptoids with a terminal cysteine residue are stored in wells of a microtiter plate as a stock (see e.g., U.S. Patent Application No. 2010/0303805, which is incorporated herein by reference), and replicate peptoid antigen arrays with individual peptoids at defined locations are printed onto maleimide-coated glass slides. For example, arrays with approximately 15,000 different octameric peptoids are tested for binding to serum-derived antibodies from normal, healthy volunteers and prostate cancer patients, respectively. Methods to screen peptoid arrays with sera and recover disease-associated peptoid antigens are described (see e.g., Reddy et al., Cell 144: 132-142, 2011 which is incorporated herein by reference). For example, antibodies bound to peptoids on the array are detected with fluorescently labeled anti-Ig antibodies (e.g., Alexa-647 labeled anti-human-IgG antibody available from Invitrogen, Carlsbad, Calif.). Arrays with bound antibodies are analyzed with a scanner at 10 μm resolution (e.g., GenePix Autoloader 4200AL Scanner available from Molecular Devices, Sunnyvale, Calif.) and scanned images are analyzed with software (e.g., GenePix Pro 6.0 available from Axon Instruments, Union City, Calif.). Peptoid antigens on the array that bind to antibodies from prostate cancer patient sera, but not to antibodies from healthy donor sera, are identified as prostate cancer disease-associated peptoid antigens, and the bound antibodies are identified as disease-associated antibodies. Disease-associated peptoid antigens are recovered from the array, analyzed and used to clone and express disease-associated monoclonal antibodies.

Differential screening strategies using the peptoid antigen array are designed to preferentially identify valuable antibodies for therapy. For example, sera from stage I prostate cancer patients are compared to healthy donor sera and to sera from stage IV patients to identify therapeutic antibodies or targets associated with progression or metastasis of prostate cancers. Differential screening of prostate cancer sera before, during and after therapy with anticancer drugs may identify therapeutic antibodies or targets associated with an antitumor immune response. For example, sera from prostate cancer patients responding to activated cell therapy such as Provenge (see e.g., Small et al., J. Clin. Onc. 24: 3089-94, 2006 which is incorporated herein by reference) can be differentially screened versus nonresponding patient's sera, or untreated patient's sera, to identify therapeutic antibodies or targets associated with an anti-cancer immune response.

To produce disease-associated monoclonal antibodies for therapy, prophylaxis, or target identification of prostate cancer, disease-associated peptoid antigens are used to isolate B cells expressing presumptive disease-associated monoclonal antibodies, and the corresponding Ig genes are amplified, cloned and expressed. Peptoid antigens identified using prostate cancer patient sera (as described above) are recovered from the array and their mass is determined using tandem mass spectrometry. Methods and instrumentation for mass spectrometry are available from Bruker Daltonics Inc., Billerica, Mass. Disease-associated soluble peptoids are resynthesized in microgram quantities using a peptide synthesizer (e.g., ABI 433A Peptide Synthesizer available from Applied Biosystems Inc., Foster City, Calif.) and the submonomer method (see e.g., U.S. Patent Application No. 2010/0303805, which is incorporated herein by reference). The peptoids are then purified by reverse phase-high pressure liquid chromatography on C18 columns (chromatography systems are available from Waters Corp., Milford, Mass.). The resynthesized, purified peptoid antigens are optionally analyzed by mass spectrometry to verify mass and then tested for binding to disease-associated antibodies as described above. The disease-associated peptoid antigens are used to screen supernatants from B cell cultures established with memory B cells from prostate cancer patients, as described.

Memory B cells are isolated from prostate cancer patients, and cultured at limiting dilution. Culture supernatants are tested for binding to disease-associated peptoid antigens. Methods for screening memory B cells with known antigens and generating monoclonal antibodies are described (see e.g., WO 2010/107939 which is incorporated herein by reference). For example approximately 30,300 CD19⁺ and surface IgG⁺ memory B cells are obtained from ten million peripheral blood mononuclear cells by cell sorting or by using magnetic beads and anti-surface IgG and anti-CD19 monoclonal antibodies. The memory B cells are seeded at approximately 1.3 cells per well in a microtiter plate and cultured with mitogens and/or activators (e.g., lipopolysaccharide, CD40 ligand, BLys) to promote antibody production. Culture supernatants are tested for binding to the disease-associated peptoids using an array displaying disease-associated peptoids and control peptoids. Methods to screen peptoid arrays with sera and recover disease-associated peptoid antigens are described (see e.g., Reddy et al., Cell 144: 132-142, 2011 which is incorporated herein by reference). For example, antibodies bound to peptoids on the array are detected with fluorescently labeled anti-Ig antibodies (e.g., Alexa-647 labeled anti-human-IgG antibody available from Invitrogen, Carlsbad, Calif.). B cell cultures producing antibodies with at least 3-fold higher fluorescence relative to control cultures and control peptoids are identified as prostate disease-associated B cells. Messenger RNA (mRNA) are obtained from the individual B cell cultures and immunoglobulin heavy (H) and light (L) chain variable (V) region genes are amplified, sequenced, cloned and expressed. Reverse transcriptase-polymerase chain reaction (RT-PCR) is used to amplify VH and VL mRNA sequences using V-region and constant (C)-region primers (see e.g., WO 2010/107939 Ibid.), and VH and VL genes are cloned in a mammalian cell expression vector containing Ig constant region genes, gamma-1 heavy chain and kappa light chain. Cloning and expression of monoclonal antibodies with expression vectors is described (see e.g., U.S. Pat. No. 7,112,439 which is incorporated herein by reference). Prostate cancer-associated monoclonal antibodies are used to prevent or treat prostate cancer and/or they are used to identify targets for prostate cancer therapy.

### PROPHETIC EXAMPLE 3

### Molecular Cloning and Expression of Disease-Associated Monoclonal Antibodies for Therapy of Multiple Sclerosis Using an Aptamer Library

Multiple sclerosis (MS) is an autoimmune disease that is characterized by IgG present in the cerebral spinal fluid (CSF), autoantibodies present in the peripheral blood, and demyelination of nerves in the central nervous system (CNS). Unknown antigen libraries comprised of aptamers are used to identify, clone and express antibodies associated with MS. Disease-associated antibodies are used for treatment or prevention of MS, as well as for identification of therapeutic targets for MS.

MS disease-associated antibodies are identified by differential screening of sera from the peripheral blood of MS patients on aptamer libraries. Sera from MS patients and healthy controls are tested for binding to aptamer libraries that may contain approximately 10⁶ unique aptamer sequences. For example a combinatorial array of aptamers containing deoxynucleotides and thio-modified deoxynucleotides (e.g., dTTP(αS), dATP(αS), dCTP(αS) and dGTP(αS)) is synthesized using phosphoramidite chemistry (using a DNA synthesizer and reagents from Applied Biosystems Inc., Foster City, Calif.) on polystyrene beads (60-70 μm diameter) with non-cleavable hexaethyleneglycol linkers (available from ChemGenes Corp., Ashland, Mass.). PCR primer sites are added to each end of the aptamer and a “pool and split” method is used to create thioaptamer libraries with a length of 52 nucleotides per aptamer and a complexity of approximately 10⁶ distinct thioaptamer sequences with one unique aptamer sequence on each bead. Methods for constructing and screening combinatorial aptamer libraries are described (see e.g., U.S. Pat. No. 7,338,762, which is incorporated herein by reference). To identify disease-associated antibodies and the aptamers to which they bind, an aptamer array containing approximately 100,000 beads (with 100,000 aptamer sequences) is incubated with fluorescently labeled IgG from MS patients, and healthy individuals, respectively. IgG is purified from MS patient sera and healthy controls using protein A sepharose columns for affinity chromatography (protein A sepharose and protocols for IgG purification are available from Sigma-Aldrich, St. Louis, Mo.). IgG from MS patients is labeled with a fluorescent dye (Cy3), and control IgG is labeled with a second dye (Cy5). Cyanine dyes (Cy3, Cy5) and protocols for conjugating them to IgG are available from Jackson ImmunoResearch Lab. Inc., West Grove, Pa. The fluorescently labeled IgGs are both allowed to bind to the aptamer sequences, and then analyzed by two color flow cytommetry to determine the ratio of Cy3 to Cy5 fluorescence. Aptamer beads with a Cy3/Cy5 ratio greater than one are collected and recovered using flow cytommetry (for example see e.g., U.S. Pat. No. 7,338,762 Ibid.). Aptamers that preferably bind IgG from MS patients are analyzed to determine their sequence using PCR. Methods to amplify aptamers from single beads using DNA primers and Taq polymerase are described (see e.g., U.S. Pat. No. 7,338,762 Ibid.). DNA fragments derived from amplified aptamers are cloned using a TA cloning kit (available from Invitrogen Inc., Carlsbad, Calif.), and sequenced using an ABI Prism 310 Genetic Analyzer (available from Applied Biosystems, Foster City, Calif.). Aptamer sequences isolated by binding to MS patient's IgG are resynthesized with thionucleotides as a single unique sequence (see above for aptamer synthesis) and retested for binding to MS patient's IgG, and control IgG, respectively. Thioaptamers which preferentially bind to MS patient's IgG (as indicated by the Cy3/Cy5 fluorescence ratio are identified as MS disease-associated aptamers which bind antibodies associated with MS. To clone the disease-associated monoclonal antibodies, the disease-associated aptamers are fluorescently labeled and used to identify B cells expressing surface IgG antibodies associated with MS.

The verified MS disease-associated aptamers are used as fluorescent probes to stain and sort cognate B cells obtained from the peripheral blood of MS patients with disease-associated antibodies in their serum. The aptamers are labeled with biotin-UTP at their 3′ end for fluorescence. A kit including reagents and methods for adding biotin-UTP to DNA is available from Pierce Biotechnology, Inc., Rockford, Ill. (see e.g., Pierce Product Sheet: “Biotin 3′ End DNA Labeling Kit,” which is incorporated herein by reference). The biotinylated aptamer is combined with B cells obtained from the peripheral blood of MS patients, and allowed to bind, then streptavidin quantum dots (Qdot 525: emission maximum near 525 nm available from Invitrogen Corp., Carlsbad, Calif.) are added to label the bound biotinylated aptamers. Methods to label mammalian cells with fluorescent aptamers are described (see e.g., Terazono et al., J. Nanobiotech. 8:8, 2010, which is incorporated herein by reference). The Qdot-aptamer labeled B cells are sorted using flow cytommetry to obtain single B cells.

Peripheral blood mononuclear cells are prepared from MS patient's blood, and B cells are enriched using anti-human IgG conjugated magnetic microbeads (magnetic beads, antibodies and protocols are available from Miltenyi Biotec, Bergisch Gladbach, Germany). Prior to single cell sorting, IgG-positive B cells are bound with biotinylated aptamers, streptavidin-Qdot525 and stained with anti-CD19-APC. Single cell sorting using a FACSVantage cell sorter (available from Becton Dickinson, Palo Alto, Calif.) collects individual Qdot-aptamer-labeled B cells in the wells of a microtiter plate containing RNA lysis buffer. Methods for single cell sorting of B cells are described (see e.g., Wardemann et al., Science 301: 1374-1377, 2003 and Wrammert et al., Nature 453: 667-671, 2008 which are incorporated herein by reference). Immunoglobulin mRNA for Immunoglobulin heavy and Immunoglobulin light chains are amplified by RT-PCR and the respective DNA sequences are determined. Molecular cloning and expression of the MS disease-associated monoclonal antibodies is done as described (see e.g., Wrammert et al., Ibid.), and the recombinant monoclonal antibodies are tested for binding to the disease-associated aptamer using the biotinylated aptamer captured on a streptavidin coated slide. Bound monoclonal antibody is detected with anti-human IgG antibodies labeled with Cy3 (available from Invitrogen Corp, Carlsbad, Calif.). Control aptamers (with scrambled nucleotide sequence) and healthy donor IgG, are included as negative controls.

MS disease-associated monoclonal antibodies can be used to treat or prevent MS directly, or the monoclonal antibodies are used to identify disease-associated antigens that represent targets for therapeutics to prevent or treat MS and/or its symptoms. Differential screening of antibodies and the corresponding B cells from MS patients in remission versus during relapse may detect valuable antibodies for therapy of MS and allow production of monoclonal antibodies for treatment, prevention and target identification.

### PROPHETIC EXAMPLE 4

### Purification of Disease-Associated Antibodies for Passive Immunization of Influenza Virus

Influenza virus-associated antibodies are identified from the sera of symptomatic or asymptomatic influenza patients using an array of unknown antigens. Patients who have recovered from infection by influenza virus are a source of antibodies which are useful for therapy of influenza viral infections (see e.g., Khurana et al., PLoS Med. 6: e1000049, 2009, which is incorporated herein by reference). Influenza disease-associated antibodies are identified, for example, using arrays of unknown antigens. For example, libraries of peptoid antigens (N-substituted oligoglycines) are constructed that can contain greater than about 100,000 different peptoid antigens. Individual peptoids with a terminal cysteine residue are placed in wells of a microtiter plate (see e.g., U.S. Patent Application No. 2010/0303805, which is incorporated herein by reference), and replicate peptoid antigen arrays with individual peptoids at defined locations are printed onto maleimide-coated glass slides. For example, arrays with approximately 15,000 different octameric peptoids are tested for binding to serum-derived antibodies from normal, healthy volunteers or influenza patients, respectively. Methods to screen peptoid arrays with sera and recover disease-associated peptoid antigens are described (see e.g., Reddy et al., Cell 144: 132-142, 2011, which is incorporated herein by reference). Antibodies bound to peptoids on the array are detected with fluorescently labeled anti-Ig antibodies (e.g., Alexa-647 labeled anti-human-IgG antibody available from Invitrogen, Carlsbad, Calif.). Arrays with bound antibodies are analyzed with a scanner at 10 μm resolution (e.g., GenePix Autoloader 4200AL Scanner available from Molecular Devices, Sunnyvale, Calif.), and scanned images are analyzed with software (e.g., GenePix Pro 6.0 available from Axon Instruments, Union City, Calif.). Peptoid antigens on the array that bind to antibodies from influenza patient sera, but not to antibodies from healthy donor sera, are identified as influenza virus disease-associated peptoid antigens, and the bound antibodies are identified as influenza disease-associated antibodies. Differential screening strategies using a peptoid antigen array are designed to preferentially identify valuable antibodies for passive immunization. For example, antibodies that recognize multiple subtypes of influenza virus are described (see e.g., Ekiert et al., Science 324: 246-251, 2009, which is incorporated herein by reference). Sera from individuals known to have recovered from different strains of influenza virus (e.g., H1N1, H3N2, and H5N1) are used to identify antibodies that recognize the same peptoid antigen, and conversely, the peptoid antigen(s) are used to purify antibodies useful for preventing multiple strains of influenza.

Peptoid antigens identified using influenza patient sera, as described above, are recovered from the array and their respective masses are determined using tandem mass spectrometry. Methods and instrumentation for mass spectrometry are available from Bruker Daltonics Inc., Billerica, Mass. Disease-associated soluble peptoids are resynthesized in milligram quantities using a peptide synthesizer (e.g., ABI 433A Peptide Synthesizer available from Applied Biosystems Inc., Foster City, Calif.), and the submonomer method (see e.g., U.S. Patent Application No. 2010/0303805, which is incorporated herein by reference). The peptoids are then purified by reverse phase-high pressure liquid chromatography on C18 columns (chromatography systems are available from Waters Corp., Milford, Mass.). The re-synthesized, purified peptoid antigens are optionally reanalyzed by mass spectrometry to verify mass, and then tested for binding to disease-associated antibodies as described above. The disease-associated peptoid antigens are used to create an affinity matrix for purification of disease-associated antibodies from influenza patient sera. For example, the disease-associated peptoid is covalently coupled to a chromatography resin (e.g., sulfhydryl coupling resin available from G Biosciences, St. Louis, Mo.) using the carboxy-terminal sulfhydryl group of the peptoid (see e.g., G Biosciences Protocol: “Sulfhydryl Coupling Resin” which is incorporated herein by reference), and the peptoid-affinity matrix is used to create a chromatography column. Prior to chromatography on peptoid-affinity columns, IgG antibodies from influenza patient sera are isolated using protein A Sepharose columns (available from Sigma-Aldrich, St. Louis, Mo.). IgG antibodies are then applied to the peptoid-affinity column. The column is washed with a neutral pH buffer, e.g., phosphate buffered saline, pH 7.4 to elute nonbinding antibodies. Then antibodies bound to peptoid are eluted with an acidic buffer (e.g., 100 mM glycine, pH 3.0), and collected into a neutral buffer (e.g., TrisHCl, pH 7.8).

Purified influenza disease-associated antibodies derived from multiple donors and/or multiple draws of an individual donor are pooled and characterized with respect to their specificity for various strains of influenza virus, (see e.g., Wrammert et al., Ibid.) and their functional activity (e.g., virus neutralization; see above). True influenza disease-associated antibodies purified on a peptoid affinity matrix are used for passive immunization of individuals at increased risk from influenza infection, such as the elderly or young children. Influenza disease-associated antibodies are stored frozen in preparation for a future influenza pandemic.

### PROPHETIC EXAMPLE 5

### Identification and Cloning of Prostate Cancer Disease-Associated Antigens Using Disease-Associated Monoclonal Antibodies and Disease-Associated Peptoid Antigens

A panel of disease-associated monoclonal antibodies is produced using sera and B cells from prostate cancer patients and unknown antigens (e.g., peptoid antigens). See, for example, Prophetic Example 2. The monoclonal antibodies are used to screen normal tissues, prostate tumor sections, prostate tumor cells, body fluids and recombinant DNA protein expression libraries, for prostate cancer disease-associated antigens.

Presumptive prostate cancer disease-associated monoclonal antibodies are used to identify the disease-associated antigens recognized by the antibodies. The monoclonal antibodies are used to establish the tissue or body fluid containing the disease-associated antigen(s). Presumptive prostate cancer-associated monoclonal antibodies may recognize antigens expressed by prostate tumor cells, normal prostate cells, or other normal cells (e.g., hematopoietic cells, vasculature cells, connective tissue cells). Moreover, disease-associated antigens may be intracellular, in the nucleus, in the cytoplasm, on the cell surface or extracellular (see e.g., Wang et al., New Engl. J. Med. 353: 1224-1235, 2005 which is incorporated herein by reference). To establish the tissue (or fluid) of origin of the presumptive prostate-disease associated antigens, the monoclonal antibodies are each tested on multi-tissue slides using immunohistochemistry. Glass slides containing fixed and frozen sections of normal human tissues and tumor cell specimens (e.g., prostate tumor cells) are available from Zyagen, San Diego, Calif. and Alpha Diagnostic, San Antonio, Tex. Procedures and reagents for detecting monoclonal antibody binding on tissue sections are given in the protocol: “Immunochemistry Procedures” from Sigma-Aldrich Co., St. Louis, Mo., which is incorporated herein by reference. For example, multi-tissue slides and negative control slides are reacted with a presumptive disease-associated monoclonal antibody and then a biotinylated secondary antibody (e.g., biotin-anti-human IgG) and ExtrAvidin peroxidase (both are available from Sigma-Aldrich Co., St. Louis, Mo.) are added to detect monoclonal antibodies bound to the tissue sections. Methods and reagents for detecting bound ExtrAvidin peroxidase are provided in “Immunochemistry Procedures” Ibid.

To localize disease-associated antigens present in body fluids (e.g., serum, lymph, cerebrospinal fluid, semen, urine, etc.), each monoclonal antibody is tested using protein arrays with biomolecules from the body fluid(s). Methods, slides, reagents and protocols for immobilizing monoclonal antibodies and testing them with serum and other fluids are available from Whatman Inc., Piscataway, N.J. (see e.g., “The FAST Guide to Protein Arrays” which is incorporated herein by reference.) For example, slides made with immobilized monoclonal antibodies at approximately 1000 μg/ml and spots approximately 110 μm in diameter are used to detect proteins in serum. Serum proteins are indirectly labeled with biotin-ULS using a Whatman Two Color Labeling and Detection Kit (available from Whatman Inc., Piscataway, N.J.; see Product Sheet: “Two Color Labeling and Detection System,” which is incorporated herein by reference). Serum proteins that bind to immobilized monoclonal antibodies are detected with a streptavidin-DY647 fluorophore conjugate using an Axon Gene Pix 4100A fluorescent micro-scanner (available from Molecular Devices, Sunnyvale, Calif.).

Immunochemistry using a disease-associated monoclonal antibody may identify prostate tumor cells that contain a disease-associated antigen. The prostate tumor cells are used to construct a complementary DNA (cDNA) expression array in a mammalian cell expression vector. For example, messenger RNAs (mRNA) are obtained from a prostate tumor cell line (e.g., PC-3 available from ATCC, Manassas, Va.) and cloned as cDNA in a viral expression vector, e.g., recombinant Sindbis virus, to create a prostate tumor cell cDNA expression array. The viral cDNA array is used to infect BHK-21 cells (available from ATCC, Manassas, Va.), which are screened for a disease-associated antigen with the corresponding disease-associated monoclonal antibodies. Methods to construct and screen viral cDNA expression libraries are described (see e.g., Koller et al., Nature Biotechnology 10: 851-855, 2001, which is incorporated herein by reference). BHK-21 cells are infected with recombinant viral particles and then screened using disease-associated monoclonal antibodies in a “plaque lift array”. Infected BHK-21 plaques that bind the monoclonal antibodies are isolated and used to prepare cDNA which are amplified using the polymerase chain reaction (PCR) and primers designed for the viral expression vector (see Koller et al., Ibid. for detailed methods). The PCR-amplified cDNA encoding the disease-associated antigen is cloned in a plasmid vector, e.g., pGEM-T available from Clontech, Palo Alto, Calif.). DNA sequence of the clone disease-associated cDNA is determined using a DNA sequencer (e.g., 3500 Genetic Analyzer available from Applied Biosystems, Foster City, Calif.). The disease-associated antigen is verified by sequence alignment and homology determinations with known human genes. Moreover the cloned cDNA is expressed in BHK-21 cells and the disease-associated monoclonal antibody is used to test for the disease-associated antigen.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
contacting a mimotope array with at least one biological tissue of a subject including one or more antibodies;
identifying at least one antibody from the at least one biological tissue corresponding to one or more antibodies bound to the mimotope array; and
isolating from the subject at least one B cell corresponding to the at least one identified antibody.

2. The method of claim 1, wherein the at least one B cell is isolated based on binding of the B cell receptor or corresponding antibody to the mimotope array.

3. The method of claim 1, wherein the mimotope array includes at least one mimotope including at least one of a peptoid, non-natural amino acid, or aptamer.

4. The method of claim 3, wherein the at least one mimotope includes a synthetic or artificial construct.

5. The method of claim 1, wherein the subject displays at least one disease symptom at the time of testing.

6. The method of claim 1, wherein the subject was recently vaccinated.

7. The method of claim 1, further comprising recording in at least one medium at least one characteristic of the mimotope array binding of at least one antibody from the biological tissue of the subject.

8. The method of claim 7, wherein the recording occurs for at least two time points.

9. The method of claim 7, further comprising predicting at least one mimotope binding based on the recorded differences.

10. The method of claim 1, wherein at least one mimotope of the mimotope array includes one or more subsets of mimotopes.

11. The method of claim 1, further comprising obtaining at least one of the proteomic or genetic sequence of at least a portion of the B cell receptor of the at least one isolated B cell.

12. The method of claim 11, further comprising synthesizing one or more antibodies based on the proteomic or genetic sequence of at least a portion of the B cell receptor.

13. The method of claim 12, further comprising identifying at least one cognate antigen of the B cell receptor.

14. The method of claim 13, wherein synthesizing one or more antibodies includes synthesizing one or more of a synthetic antibody, artificial antibody, antibody mimetic, recognition element mimetic, or other antibody.

15. The method of claim 13, wherein synthesizing the one or more antibodies includes at least one of de novo synthesis of one or more antibodies, or isolating one or more antibodies from an expression system.

16. The method of claim 13, further comprising providing at least one of the one or more antibodies to the subject.

17. The method of claim 1, further comprising isolating the at least one identified antibody.

18. The method of claim 17, further comprising providing the at least one isolated antibody to the subject.

19. The method of claim 17, further comprising identifying at least one cognate antigen of the at least one identified antibody.

20. The method of claim 19, further comprising developing a vaccine based on the at least one cognate antigen.

21. The method of claim 17, further, comprising contacting the at least one identified antibody with a mimotope array.

22. The method of claim 21, further comprising analyzing binding of the at least one identified antibody with the mimotope array.

23. The method of claim 22, further comprising correlating the binding of the at least one identified antibody with at least one health status.

24. The method of claim 1, further comprising manipulating the at least one isolated B cell.

25. The method of claim 24, wherein the manipulating the at least one isolated B cell includes at least one of inducing the at least one B cell to proliferate, inducing the at least one B cell to differentiate, inducing the at least one B cell to release at least one of an antibody or cytokine, or inducing attachment of the at least one B cell to a substrate.

26. The method of claim 1, wherein the biological tissue includes at least one biological fluid.

27. The method of claim 26, wherein the at least one biological tissue includes at least one of blood, serum, plasma, saliva, bronchial lavage, buccal swab, ascites, urine, milk, lacrimal secretions, sweat, semen, vaginal secretions, tumor biopsy, bile, or other biological fluid.

28. The method of claim 1, wherein one or more steps are performed by a computing device.

29. The method of claim 28, further comprising generating at least one output to a user.

30. The method of claim 29, wherein the at least one output includes at least one of a mimotope array location of binding of one or more antibodies, identification of the structure of at least one mimotope that has binding of one or more antibodies, or structure of at least one predicted antigen based on mimotope binding.

31. The method of claim 29, wherein the at least one output occurs in real-time.

32. The method of claim 29, wherein the user includes at least one entity.

33. The method of claim 32, wherein the entity includes at least one person or computer.

34. The method of claim 29, wherein the at least one output includes at least one output to a user readable display.

35. The method of claim 34, wherein the user readable display includes at least one human readable display.

36. The method of claim 34, wherein the user readable display includes one or more active displays.

37. The method of claim 34, wherein the user readable display includes one or more passive displays.

38. The method of claim 34, wherein the user readable display includes one or more of a numeric format, graphical format, or audio format.

39. A method comprising:
contacting a first mimotope array with at least one biological tissue of a first subject;
isolating at least one antibody having the ability to bind the at least one mimotope associated with at least one disease symptom.

40. The method of claim 39, further comprising storing separately each antibody having the ability to bind the at least one mimotope associated with the at least one disease symptom.

41. The method of claim 39, wherein the mimotope array includes at least one mimotope including at least one of a peptoid, non-natural amino acid, or aptamer.

42. The method of claim 41, wherein the at least one mimotope includes a synthetic or artificial construct.

43. The method of claim 39, wherein one or more steps are performed by a computing device.

44. The method of claim 39, further comprising generating at least one output to a user.

45. The method of claim 44, wherein the at least one output includes at least one of a mimotope array location of binding of one or more antibodies, identification of the structure of at least one mimotope that has binding of one or more antibodies, or structure of at least one predicted antigen based on mimotope binding.

46. The method of claim 44, wherein the at least one output occurs in real-time.

47. The method of claim 44, wherein the user includes at least one entity.

48. The method of claim 47, wherein the entity includes at least one person or computer.

49. The method of claim 44, wherein the at least one output includes at least one output to a user readable display.

50. The method of claim 49, wherein the user readable display includes at least one human readable display.

51. The method of claim 49, wherein the user readable display includes one or more active displays.

52. The method of claim 49, wherein the user readable display includes one or more passive displays.

53. The method of claim 49, wherein the user readable display includes one or more of a numeric format, graphical format, or audio format.
